(19) United States
(12) Patent Application Publication
(10) Pub. No.: US 2021/0353892 A1
(43) Pub. Date: Nov. 18, 2021

(54) SYSTEM FOR SUPPLYING GASES FOR VENTILATION AND OXYGENATION WITH FEED OF INHALABLE SUBSTANCES

(71) Applicant: Drägerwerk AG & Co. KGaA, Lübeck (DE)

(72) Inventors: Henning GERDER, Lübeck (DE); Christian BRENDLE, Lübeck (DE); Marco MONNIG, Lübeck (DE)

(21) Appl. No.: 17/318,281

(22) Filed: May 12, 2021

(30) Foreign Application Priority Data
May 13, 2020 (DE) 10 2020 112 951.3
Jan. 6, 2021 (DE) 10 2021 100 091.2

(51) Int. Cl. A61M 16/06; A61M 16/22; A61M 16/08; A61M 16/20; A61M 16/16; A61B 5/00; A61B 5/08

(52) U.S. Cl. CPC A61M 16/0672; A61M 16/22; A61M 16/0891; A61M 16/205; A61M 16/16; A61M 2205/75; A61B 5/0044; A61B 5/08; A61M 2202/0225; A61M 2202/0241; A61M 2202/0208; A61B 5/0036

(57) ABSTRACT

A system (1000) feeds substances to a patient (30) with a ventilation of the patient and with an oxygenation of the patient. The system (1000) has at least one ventilation system (1), a sedation by inhalation system (17) with a dispensing system (7), an oxygenation system (2), a breathing gas dispensing path (3), a purge gas dispensing path (4), a breathing gas connection system (5), a connection element (25) located adjacent to the patient, an oxygenation connection system (6) and a switching unit (8). The switching unit (8) is configured to distribute and to split a quantity of an inhalable substance dispensed into a gas mixture by means of the dispensing system (7) between the connection element (25) located adjacent to the patient and the oxygenation system (2). At least one control unit (9, 10, 11, 12) is configured to control the switching unit (8) and/or the system (1000).

### CROSS REFERENCE TO RELATED APPLICATIONS

This application claims the benefit of priority under 35 U.S.C. § 119 of German Applications 10 2020 112 951.3, filed May 13, 2020, and 10 2021 100 091.2, filed Jan. 6, 2021, the entire contents of which are incorporated herein by reference.

### TECHNICAL FIELD

The present invention pertains to a system for supplying gases for ventilation and oxygenation with feed of inhalable substances. A combined system with a device for supplying breathing gases, gases or gas mixtures for the ventilation and oxygenation of a patient with gases or gas mixtures and with a device for an extracorporeal membrane oxygenation of a patient is described. The system according to the present invention with the devices for ventilation and extracorporeal membrane oxygenation makes it possible to feed inhalable substances or anesthetics by means of the gas or gas mixture supplied to the patient. The substances are, for example and preferably, gases dissolved in the gas phase or in the vapor phase, for example, anesthetics, anesthetic gases, narcotics, drug active ingredients or drugs dissolved in the gas phase or in the vapor phase, which are suitable for administration by inhalation into the breathing gas. The term “breathing gas” is defined below in the sense of the present invention as a generic term for quantities of gas fed to the patient or removed from the patient, so that it is defined as inhaled gas, exhaled gas, breathing gases, inhaled gases, exhaled gases as well as breathing gas, breathing gases.

### TECHNICAL BACKGROUND

An application of conventional ventilation in intensive care units as well as during the carrying out of surgery often lead to undesired side effects, for example, barotrauma/volutrauma and aspiration, which may cause damage to the lungs in some cases and complications such as pneumonia or sepsis. To avoid further damage and as a treatment in case of damage to the heart or lungs, there are approaches to oxygenation and circulatory support, such as venovenous extracorporeal membrane oxygenation (v.v. ECMO), pumpless extracorporeal lung assist (pECLA) and veno-arterial extracorporeal membrane oxygenation (v.a. ECMO).

Ventilators and anesthesia devices which can be used for ventilation in an intensive care unit (ICU) or for performing surgical procedures in operating rooms (OR) are known from the state of the art.

US 2016067434 A1 discloses a ventilator for ventilating a patient for use in an intensive care unit. The ventilator shown shall serve the purpose of avoiding complications during the performance of the ventilation.

U.S. Pat. No. 4,148,312 A discloses a combination of an anesthesia device and a ventilator. Unconsciousness, insensitivity to pain and relaxing of muscles of the patient are essential for the performance of ventilation. Different volatile anesthetics (halothane, isoflurane, desflurane, sevoflurane, ether) as well as nitrous oxide with different hypnotic, analgesic and muscle-relaxing properties in combination with air and oxygen are fed to this end to the patient by inhalation by means of an endotracheal tube. In addition, drugs are usually also administered into the blood circulation in an invasive manner. The administration of the volatile anesthetics or narcotics into the breathing gas or into the breathing gas mixture may be carried out, for example, by means of evaporation with an anesthetic evaporator, also called anesthetic vaporizer or vaporizer.

US 2016008567 A1 discloses a system for dispensing narcotics or volatile anesthetics.

WO 09033462 A1 discloses an anesthetic evaporator with a storage tank and with a feeding and dispensing device, wherein a vapor pressure of the anesthetic is generated by an elevated temperature and a saturated anesthetic vapor is generated.

So-called heart-lung machines (HLM) are used especially when performing surgical procedures on the heart. These heart-lung machines (HLM) assume the function of the heart and lungs, i.e., the feeding of oxygen into the blood circulation of the patient and the removal of carbon dioxide from the blood circulation of the patient as well as the flow of blood into the blood vessels during the surgical procedure. Thus, for example, GB 2568813 A1 discloses a heart-lung machine for an extracorporeal gas exchange and oxygenation.

WO 2009033462 A1 discloses a device for supplying anesthetic gas. The device makes it possible to supply saturated anesthetic vapor and to feed it to a patient without the need for external fresh gas or carrier gas.

US 2020038564 A1 discloses a blood pump, which is suitable for the extracorporeal transport of blood.

U.S. Pat. No. 9,901,885 B2 discloses a membrane, which is configured and intended for a blood-to-gas and gas-to blood exchange.

U.S. Pat. Nos. 6,174,728 B1, 4,279,775 A and US 2003064525 A disclose devices for determining components and blood gases in the blood of living beings.

### SUMMARY

With the knowledge of the above state of the art, an object of the present invention is to provide a system, which makes possible the feeding of substances in the gas form into the breathing circuit and the feeding of substances in the gas form into the blood circulation of a patient outside the body.

Another object of the present invention is to provide a device, which makes possible the distribution and/or splitting of partial quantities of breathing gas into the breathing circuit and into the blood circulation of a patient.

The above objects are accomplished by the ventilating and oxygenating system according to the invention and by the gas splitting unit according to the invention.

The object is accomplished with respect to a system for feeding substances in the gas form by a ventilating and oxygenating system for supplying gases for the ventilation and oxygenation of a patient with the feeding of substances having features according to the invention.

The object is accomplished with respect to a distribution and/or splitting of partial quantities of breathing gas into the breathing circuit and into the blood circulation of a patient by a gas splitting unit having features according to the invention.

Advantageous embodiments of the present invention appear from the disclosure and will be explained in more detail in the following description partly with reference to the figures.

A first inventive aspect is formed by a system for feeding substances with a feed in the form of a gas both into the breathing circuit as well as into the blood circulation of a patient. The system according to the present invention for feeding substances makes possible a feeding of substances in the form of a gas into the breathing circuit and a feeding of the substances in the form of a gas outside the body into the blood circulation of a patient. Thus, it makes possible, for example, for an application in a clinical setting in an intensive care unit (ICU), a simultaneous and/or parallel, coordinated operation with feeding of substances for a sedation by inhalation into the blood circulation as well as into the breathing circuit. The feeding of substances for sedation by inhalation may be carried out by means of the simultaneous and/or parallel, coordinated operation via access via the airways of the patient as well as via a gas/blood exchange system (membrane oxygenation, ECMO, oxygenator, oxygenation system). Both an administration of substances for a sedation by inhalation via the lungs into the cardiovascular system of the patient and an administration of these substances via the gas/blood exchange system into the blood circulation of the patient (extracorporeal circulation) may be carried out at the same time with the system according to the present invention.

A system according to the present invention has:
a ventilator with a ventilation system (VS) and
with a breathing gas connection system,
with a connection element (Y-piece) located close to the patient,
an oxygenation system (OS) with an oxygenation connection system,
a sedation by inhalation system (SIS) with a dispensing system (DS),
with a gas removal port for inhaled gas,
with a reflection unit (CR),
with a gas return port for inhaled gas,
a switching unit,
a breathing gas dispensing path,
a purge gas dispensing path and
at least one control unit.

The ventilation system is configured to supply breathing gases to the patient. The breathing gas connection system is configured for a gas-carrying connection for a supply with feeding and removal of quantities of breathing gases to and from the patient. The sedation by inhalation system (SIS) is formed by the dispensing system, the gas removal port for removing partial quantities of breathing gas from the inhaled gas, the reflection unit and the gas return port for returning the partial quantities of breathing gas from the dispensing system to the switching unit.

The sedation by inhalation system (SIS) is configured with the dispensing system for dispensing inhalable substances. Partial quantities of breathing gas are made available and fed to the sedation by inhalation system (SIS) from the ventilator or from the ventilation system.

A partial quantity of breathing gas enriched with inhalable substances is fed and made available to the switching unit by the sedation by inhalation system (SIS) by means of the gas removal port and by the breathing gas connection system.

The switching unit makes possible according to the present invention a splitting and/or distribution of quantities of gas enriched with inhalable substances into the breathing gas dispensing path and into the purge gas dispensing path.

A partial quantity of breathing gas enriched with inhalable substances is fed and made available by the switching unit for the airways of the patient by the switching unit by means of the breathing gas dispensing path and by means of the connection element located close to the patient.

A partial quantity of breathing gas enriched with inhalable substances is fed and made available by the switching unit by means of the purge gas dispensing path and by means of the oxygenation system.

A partial quantity of breathing gas enriched with inhalable substances is fed and made available to the patient by the switching unit by means of the breathing gas connection system via the connection element located close to the patient.

An additional partial quantity of breathing gas not enriched with inhalable substances is fed and made available to the patient by means of the breathing gas connection system via the connection element located close to the patient.

Quantities of blood enriched with inhalable substances are fed to the patient by the oxygenation system by means of a purge gas flowing in the purge gas dispensing path via the oxygenation connection system. A gas-to-blood exchange as well as a blood-to-gas exchange take place to this end in the oxygenation system, wherein quantities of gas supplied and enriched with oxygen O₂ and with the inhalable substances enter a blood circulation of the patient from the oxygenation connection system while flowing around a membrane and quantities of carbon dioxide CO₂, produced by the metabolism of the patient, pass at the same time over from the blood circulation of the patient into the purge gas dispensing path. These processes of the gas-to-blood and blood-to-gas exchange are called oxygenation and decarboxylation.

The oxygenation connection system is configured for a fluidic connection to the blood circulation for feeding and removing quantities of blood of the patient. The blood supplied by the oxygenation system and enriched with oxygen is fed to the patient as a quantity of fresh and oxygen-enriched blood invasively with a supply line by means of the oxygenation connection system.

A quantity of blood enriched with carbon dioxide is returned from the patient by means of the oxygenation connection system to the oxygenation system. The oxygenation connection system thus makes it possible to supply the patient with quantities of blood enriched with volatile substances and with oxygen (O₂) and to remove quantities of blood enriched with carbon dioxide (CO₂).

In the usual embodiment, the ventilation system is part of a ventilator. Ventilation systems for ventilators usually have suitable devices for supplying, feeding and removing breathing gases and substances to and from the patient, e.g., devices for mixing gases and for feeding gases, for example, at least one gas feed unit (blower, piston drive, valve array), as well as devices for carrying gas, such as breathing gas connection system, for example, in the form of ventilation tubes and of a connection element, the so-called Y-piece, for connecting the ventilation tubes to an endotracheal tube, to a breathing mask or to a tracheostoma. In addition, connection elements that also comprise an exhalation valve located close to the patient are known as well. In addition to the ventilation system, ventilators also have elements, especially sensors, for the detection by measurement of given and/or set pressures, flow rates and other operating parameters of a mechanical ventilation with feed of gases and gas mixtures. At least the following parameters are set and/or monitored for the mechanical ventilation: Inspiratory and expiratory ventilation pressures, ventilation rate, inhalation to exhalation ratio, pressure upper and lower limits, flow rate upper and lower limits, volume upper and lower limits and gas concentrations. In the sense of the present invention, the ventilator and the ventilation system support the system for feeding substances in terms of the object and function of ensuring ventilation of the lungs, i.e., to ensure the collapse of the lungs or of individual regions (alveoli) of the lungs. In addition, the ventilation system supports the patient in the O₂/CO₂ gas exchange in the lungs. The system for feeding substances has suitable elements for removing, feeding and/or returning quantities of breathing gases. The breathing gas supplied by the ventilation system is fed to the patient as fresh breathing gas with an inspiratory ventilation tube by means of an inspiratory path of the breathing gas connection system. The breathing gas or breathing gas mixture exhaled by the patient is returned or removed by means of the breathing gas connection system.

The inspiratory path and the expiratory path of the breathing gas connection system are merged close to the patient usually by means of a connection element located close to the patient, a so-called Y-piece, at the location of the patient. The air exhaled by the patient enters into the environment or into a suitable system for receiving used quantities of gas via an expiratory path of the breathing gas connection system by means of an exhalation valve, the so-called exhalation valve, often called EX valve. Depending on the configuration of the ventilator, the exhalation valve may be arranged in the ventilator itself, so that exhaled breathing gases can flow into the environment by means of a tracheostoma, an endotracheal tube or nasal mask via the connection element (Y-piece) and an expiratory ventilation tube and the exhalation valve. In an alternative embodiment, the exhalation valve may be arranged outside the ventilator close to the patient, so that exhaled breathing gases can flow directly into the environment by means of a tracheostoma, endotracheal tube or nasal mask via the exhalation valve. No return of breathing gases with an expiratory ventilation tube into the ventilator via the breathing gas connection system is intended in such a configuration.

The sedation by inhalation system (SIS) makes it possible to supply inhalable substances, for example, substances with hypnotic properties or effects (narcotics), or with analgesic or muscle-relaxing properties or effects. The sedation by inhalation system (SIS) makes it possible to dispense or add the inhalable substances via the switching unit into the ventilation system and/or into the oxygenation system and in this manner it makes available quantities of gas enriched with the inhalable substances for the breathing circuit and for the lungs of the patient and/or for the blood circulation of the patient via the oxygenation system.

The connection between the switching unit with the connection element located close to the patient with feed of the partial quantity of breathing gas enriched with the inhalable substances and the patient is brought about by means of the breathing gas dispensing path.

The connection between the switching unit and the oxygenation system with the feed of the partial quantity of breathing gas enriched with the inhalable substances to the oxygenation system is brought about by means of the purge gas dispensing path.

The at least one control unit is configured according to the present invention for controlling the switching unit. The control comprises here a coordination of the splitting and/or distribution of quantities of gas supplied and/or fed by the dispensing system between the dispensing paths, i.e., between the purge gas dispensing path and the breathing gas dispensing path. The control unit carries out, so to speak, a gas or gas mixture management between the two dispensing paths. The quantities of gas made available and/or supplied by the sedation by inhalation system (SIS) and/or by the dispensing system are enriched or saturated with quantities of substances, with quantities of volatile substances or with quantities of volatile anesthetics. With the control and/or coordination of the gas or gas mixture management, the at least one control unit sets suitable specifications as to quantities of substances, volatile substances or volatile anesthetics to be added via the breathing gas connection system to the quantities of breathing gas fed into the breathing circuit and into the lungs of the patients or into the oxygenation system and hence then into the blood circulation of the patient from the oxygenation system via the oxygenation connection system with fed or exchanged blood.

A balance can be set up when feeding inhalable substances, for example, between an inhalation anesthesia and an extracorporeal anesthesia, and this balance can also be eliminated during the treatment for medical reasons by controlling and coordinating the switching unit by means of the at least one control unit, especially by means of the control unit of the switching unit. The at least one control unit can thus implement in practice specifications of a user concerning the setting or changing of a main focus of the treatment in relation to the balance between extracorporeal sedation by means of the oxygenation or sedation by inhalation by means of the sedation by inhalation system (SIS) during the operation of the system according to the present invention, which makes it possible to feed substances in the gas form into the breathing circuit and a feed of substances in the gas form outside the body into the blood circulation of a patient.

The sedation by inhalation system (SIS) has, for example, a gas removal port for inhaled gas for removing a partial quantity of inhaled gas from the inspiratory path of the breathing gas connection system as a suitable element for feeding quantities of breathing gases to the dispensing system.

The sedation by inhalation system (SIS) has, for example, a gas return port for inhaled gas for returning partial quantities of inhaled gas from the switching unit back into the inspiratory path of the breathing gas connection system and/or to the connection element located close to the patient (Y-piece) as a suitable element for feeding quantities of breathing gases to the patient. The sedation by inhalation system (SIS) has suitable elements for storing and/or supplying quantities of exhaled breathing gases or breathing gas mixture from the patient. The sedation by inhalation system (SIS) has, for example, a reflection unit or an anesthetic gas reflector as a suitable element. The reflector may be configured, for example, as a carbon reflector, which is configured for the storage or buffering of the inhalable substances over a period of several days. An exemplary embodiment of a reflection unit has in the gas stream for the breathing gas a chamber with a reflection agent, for example, granules of a suitably impregnated activated carbon. The activated carbon makes it possible to adsorb, buffer and bind for a short time the inhalable substances from the exhaled gas of the patient during the exhalation by the patient and to release the inhalable substances with the subsequent inhalation of the inhaled gas back into the breathing gas connection system with feeding to the patient.

Such a reflection unit is arranged in or at the breathing gas connection system, preferably at the connection element (Y-piece) or close to the connection element (Y-piece). As an alternative, the reflection unit may be configured as a part of the connection element (Y-piece) of the breathing gas connection system. As an alternative, the connection element (Y-piece) may be configured as a part of the reflection unit in the breathing gas connection system. The reflection unit has elements for filtering and/or buffering gas components and/or substances, especially inhalable and/or volatile substances, in the breathing gas. The reflection unit is also called at times reflector, gas reflector or also anesthetic gas reflector.

The dispensing system may be configured in a preferred embodiment of the system for dispensing inhalable and/or volatile substances or volatile anesthetics.

An additional chamber, which absorbs, buffers for a short time and binds moisture present in the exhaled gas during the exhalation and then makes possible a feeding of humidified inhaled gas to the patient during the subsequent inhalation, may be arranged in the reflection unit in a preferred embodiment. Such an additional chamber arranged in the gas stream thus has the function of a filter element in the form of a so-called HME (Heat and Moisture Exchanging) filter.

In another preferred embodiment, an additional filter element, which is configured for filtering with retention of germs, viruses or bacteria in the breathing gas, may be arranged in or at the reflection unit in the additional chamber or in another, additional chamber in another preferred embodiment.

The gas return port may be configured as a component of the connection element located close to the patient. The gas return port may be configured as a component of the reflection unit.

The gas return port, the reflection unit and the connection element located close to the patient may be configured as one assembly unit in a preferred embodiment. The gas return port, the connection element located close to the patient and the reflection unit may be configured in a combination with the additional filter element and/or with the HME filter as one assembly unit in a preferred embodiment.

In a preferred embodiment, the gas removal port as well as the gas return port for inhaled gas may be configured in a common assembly unit with the reflection unit and/or with the breathing gas connection system and/or with the connection element located close to the patient (Y-piece) and/or with the additional filter element and/or with the HME filter. The gas removal port as well as the gas return port for inhaled gas may be configured in a common assembly unit with the reflection unit with an exhalation valve located close to the patient and/or with the breathing gas connection system and/or with the connection element (Y-piece) located close to the patient and/or with the additional filter element and/or with the HME filter in a preferred embodiment.

For the supply, the sedation by inhalation system (SIS) has a dispensing system with correspondingly configured elements for dispensing inhalable substances. Suitably configured elements for dispensing inhalable substances are, for example, valves or valve arrays in the form of controlled, i.e., electrically or electronically controlled or regulated valves for dispensing inhalable substances into a gas mixture. These include, for example, magnetic or electromagnetic control valves, as well as piezo valves or piezo actuators. Suitably configured elements for dispensing inhalable substances are, for example, devices for evaporating or vaporizing inhalable substances. Suitable devices for evaporating or vaporizing inhalable, preferably volatile substances are formed, for example, by anesthetic evaporators. Such anesthetic evaporators, usually called vaporizers, make it possible to enrich the gas stream with volatile substances suitable for the sedation by inhalation or sedation of a living being, for example, to increase a settable concentration of volatile anesthetics, e.g., desflurane, halothane, isoflurane, sevoflurane, and ether. Vaporizers operate according to the dispensing principle of changing flow rate ratio between a main stream and a side stream. The main stream and the side stream are merged at the outlet of the vaporizer. Consequently, saturation of the gas fed with the volatile substances is carried out in the side stream, and the degree of addition, and hence also the concentration, of the substances can be set at the outlet of the vaporizer by adjusting or setting the flow rate ratio of the main stream and the side stream. Consequently, the gas stream fed is enriched in the dispensing system with substances suitable for the sedation by inhalation or sedation of a living being.

A suitable possibility of dispensing inhalable substances by the dispensing system in the sedation by inhalation system (SIS) is made possible, for example, by branching off a partial quantity of inhaled gas from the total quantity by means of the gas removal port at the breathing gas connection system, at the connection element located close to the patient, at the reflection unit or at the ventilation system, and then to introduce a partial quantity of inhaled gas from the total quantity and then to add defined quantities of inhalable substances to this partial quantity in the dispensing system. Such a dispensing may be carried out by a dispensing valve, which dispenses the inhalable substances into the branched-off partial quantity of the inhaled gas in pulsed manner over time.

The branched-off partial quantities of the inhaled gas, which are enriched in this case with inhalable substances, are subsequently introduced again into the gas stream into the breathing gas connection system via the gas return port, preferably or as an example at the reflector or at the connection element (Y-piece) and are fed to the patient. The feeding of the volatile substances is carried out as an addition into the inhaled gas. A gas mixture of air and oxygen, made available for carrying out a ventilation treatment in terms of pressure, pressure changes over time, flow rates and volume, reaches the patient from the ventilator or from the ventilation system via an inspiratory path of the breathing gas connection system. Partial quantities of this inhaled gas are sent to the dispensing system by means of the gas removal port via a connection element usually configured as a tubing. The inhalable or volatile substances are dispensed in the dispensing system for the further use during the treatment of the patient via the lungs and/or via the blood circulation. The inhalable substances are fed to the breathing gas by means of the dispensing system for a sedation by inhalation, for example, in the form of volatile anesthetics or in the form of additional substances and they reach the switching unit via a connection element, which is usually likewise configured as a tubing.

In an alternative embodiment, the addition of the inhalable substances may be carried out by the dispensing system by a control of a variable branched-off partial quantity from the total quantity of inhaled gas, preferably in a combination with a constant addition through the dispensing valve or by a variable addition by an adjusting valve, for example, a proportional valve. If the inhalable substances are in the liquid form, a device for warming, for example, heating, which makes possible the conversion of the inhalable substances from the liquid phase into a gas phase, is provided in the sedation by inhalation system (SIS), so that an addition of inhalable substances to the inhaled gas in the gas form can be carried out by the adjusting valves or dispensing valves.

The control unit may be configured in a preferred embodiment to control the dispensing of the inhalable substances, configured as an addition pulsed over time or configured as a constant or variable addition, and/or the control the quantity of the branched-off partial quantity by the dispensing system on the basis of concentrations of the inhalable substances, which concentrations are determined at the connection element (Y-piece), at the breathing gas connection system or at the reflection unit.

For example, a measuring system for a process gas analysis (PGA) for determining a gas concentration, especially a gas concentration of an inhalable substance or of an anesthetic gas concentration in the exhaled gas, which measuring system may be arranged as a separate measuring system in the system for feeding substances, may be associated with the system for feeding substances or may be configured as an element of the sedation by inhalation system (SIS) or as an element of the dispensing system, may be used to determine the concentrations of the inhalable substances in the inhaled gas. Such a measuring system (PGA) is configured to carry out an analysis of the breathing gas concerning the concentrations of inhalable substances, especially anesthetic concentrations or nitrous oxide (nitrous oxide, N₂O), as well as carbon dioxide CO₂ or oxygen (O₂) on the basis of a quantity of breathing gas from the breathing gas connection system or from the connection element, which quantity of breathing gas is supplied by suction-based feeding and by means of a measured gas line. The inhalable substances are preferably added here to the inhaled gas by the dispensing system during the phases of inhalation, while the concentration determinations are carried out in the exhaled gas preferably during the phases of exhalation concerning the concentrations of inhalable, inhalable volatile substances, especially anesthetic concentrations or nitrous oxide (nitrous oxide, N₂O) as well as concerning carbon dioxide CO₂ or oxygen (O₂). In particular, concentrations at the end of the phases of exhalation, so-called end-tidal concentrations of carbon dioxide (etCO₂), of at least one inhalable substance or at least one anesthetic (etVA) are of interest here for checking the ventilation and/or the dispensing of the inhalable substances in the system for feeding substances.

In a preferred embodiment, the dispensing system is configured to carry out the addition of the inhalable substances on the basis of an end-tidal concentration of at least one inhalable substance or of at least one anesthetic.

In a preferred embodiment, the dispensing system is configured to carry out the addition of the inhalable substances, which addition is pulsed over time, by means of the dispensing valve on the basis or as a function of the end-tidal concentration of an inhalable substance or of at least one anesthetic.

The dispensing system is configured in a preferred embodiment to carry out the control of a variable branched-off partial quantity of the total quantity of inhaled gas by the adjusting valve on the basis or as a function of the end-tidal concentration of an anesthetic.

The dispensing system is configured for dispensing volatile or inhalable substances and/or for dispensing volatile anesthetics. This offers, on the one hand, the advantage that it is made possible to administer volatile substances with the system for feeding substances by means of the ventilation system and of the sedation by inhalation system, so that, for example, anesthesia by inhalation can thus be carried out. However, quantities of volatile drugs may also be administered by means of the ventilation system and of the sedation by inhalation system. The dispensing system is thus configured for dispensing volatile substances, for example, drugs. The following list comprises some examples of possibilities, possibly also of drug active ingredients that are also soluble in the gas phase or in the vapor phase, of how substances or agents for influencing the cardiovascular system, e.g., drugs acting on the blood pressure and on the heart rate, drugs for influencing the metabolism, the fluid balance or the hormonal situation of the patient, as well as drugs that may be administered concerning function and/or cure or recovery or for pain treatment as therapeutic agents for organs, e.g., the lungs, heart, kidneys, pancreas, liver, stomach, intestines, sex organs, sensory organs, brain, nervous system, bronchial tract, skeleton, skin, and muscles, thyroid gland, gallbladder, can be fed by inhalation in the gas phase or in the vapor phase.

In a special embodiment, the dispensing system may also be configured as a part of the sedation by inhalation system (SIS), and the dispensing system may also be configured in another special embodiment as a part of the ventilator or also as a part of the breathing gas connection system in another special embodiment.

The advantage arises that the system for feeding substances by means of the oxygenation system makes it possible to administer volatile agents, for example, to administer volatile anesthetics or volatile drugs with hypnotic properties (narcotics) and with analgesic or muscle-relaxing properties into the blood circulation.

The switching unit makes possible according to the present invention a switching, splitting or distribution of quantities of gas and/or inhalable substances for sedation, for example, volatile anesthetics or drugs. A switching, splitting or distribution of quantities of gas is carried out according to the present invention by means of the breathing gas connection system and the connection element (Y-piece) with reflection unit, on the one hand, and the oxygenation system, on the other hand. A feeding with splitting or distribution of the inhalable or volatile substances, anesthetics or additional substances also takes place indirectly with the switching, splitting or distribution from the dispensing system into the breathing gas connection system with the connection element located close to the patient and/or to the oxygenation system. Suitable units of the switching unit for switching and distribution are, for example, valves or valve arrays, 3/2-way valves or a combination of two 2/2-way valves arranged in parallel in the gas flow with corresponding state control for distribution and splitting into partial quantities from the dispensing valve by means of the breathing gas dispensing path to the breathing system, or by means of the purge gas dispensing path to the oxygenation system. The switching unit is configured for switching between the two dispensing paths and, in interaction with the two dispensing paths, for distributing and splitting the enriched quantities of breathing gas to the oxygenation system and to the connection element located close to the patient. The inhalable or volatile substances are fed to the breathing gas by means of the dispensing system and of the sedation by inhalation system (SIS) of the switching unit and they reach from the switching unit either the breathing gas by means of the gas return port and via the breathing gas dispensing path and the breathing gas connection system, usually also via the connection element (Y-piece) located close to the patient, and the bronchial tract and the lungs of the patient by means of the endotracheal tube, the tracheostoma or the nasal mask, or the oxygenation system from the switching unit via the purge gas dispensing path and then the blood circulation of the patient from the oxygenation system by means of the oxygenation connection system. In configurations of the system for feeding substances in the area of intensive care or emergency medicine, the switching unit is arranged in the gas flow downstream of the dispensing system. The switching unit may also be configured as a part of the sedation by inhalation system (SIS in a special embodiment, and the switching unit may also be configured as a part of the ventilator in a special embodiment.

In a special embodiment, the switching unit may be configured as a part or component of the breathing gas connection system or as a part of the oxygenation connection system. In a special embodiment, the switching unit may be configured as a part of the breathing gas dispensing path or as a part of the purge gas dispensing path. The switching unit may also be configured as a part of the dispensing system in a special embodiment.

The oxygenation system is configured to supply oxygen and to remove carbon dioxide in a blood circulation leading to the patient. The oxygenation system has a membrane for a gas/blood exchange. A quantity of oxygen is fed by means of this membrane by means of a purge gas into the quantity of blood of the blood circulation of the patient and a quantity of carbon dioxide is removed from the blood circulation of the patient. The purge gas is supplied to the oxygenation system by means of the purge gas connection path from the switching unit.

The transport of the quantity of blood to the patient and from the patient may take place in a preferred embodiment by means for feeding blood, for example, by a blood feed unit (pump). Such a blood feed unit (pump) is preferably arranged in or at the oxygenation connection system or in or at the oxygenation system and is used to transport a quantity of blood to the patient and away from the patient. Such a pump may be coupled venovenously (VV-ECMO) or arteriovenously (VA-ECMO) by means of suitable infusion cannulas and tubes with typical external diameters in the range of about 3.0 mm to 12.0 mm. The pump delivers quantities of flowing blood in the range of 0.2 L/min to 10 L/min to the oxygenation system and back again. The access to the blood circulation of the patient is, for example, via the femoral artery or the femoral vein in this case as well, and, as an alternative, also via the femoral artery and the external jugular vein. The blood feed unit makes it possible, especially in an embodiment of a blood feed unit with adjustable flow rate, to carry out the extracorporeal blood gas exchange concerning the removal of carbon dioxide and the feed of oxygen such that it is individually coordinated with the situation and the patient.

In a special embodiment of the oxygenation system, the transport of the quantity of blood to the patient and from the patient may be carried out without an external feeding of blood, for example, by a pump. The transport of the quantity of blood to the patient and away from the patient is carried out in such a special embodiment by the pumping capacity of the heart of the patient himself. This is called pumpless extracorporeal membrane oxygenation or pumpless extracorporeal lung assist (pECLA). The pumpless extracorporeal membrane oxygenation is coupled arteriovenously, for example, by means of the femoral artery and the femoral vein by means of suitable infusion cannulas and tubes with typical internal diameters in the range of about 3 mm to 7 mm, so that the heart typically feeds blood outside the body at a flow rate in the range of 2 L/min to 2.5 L/min to the oxygenation system and back again.

Preferred embodiments of the system according to the present invention for feeding substances may be configurations of the control unit in the form of a central control system or of a central control unit. These additionally preferred embodiments offer the advantage that a variety of information can be processed and compared with one another centrally and the checking, control, and/or regulation of the ventilation, of the extracorporeal blood gas exchanges or of the carrying out of the treatment can be coordinated and controlled centrally. Changes in the modes of operation or treatment, for example, the setting of a balance between an anesthesia by inhalation and of an extracorporeal anesthesia, and also the elimination of this balance during the treatment for medical reasons with setting of a new main focus of the treatment on, e.g., essentially extracorporeal feed of drugs or sedative substances, anesthesia or the administration of drugs or sedative substances by inhalation, can be coordinated centrally.

However, preferred embodiments of the system according to the present invention for feeding substances may also be configured with a plurality of individual control units, which will then form a common control of the system with one another. The control of the system may likewise be configured by means of a plurality of control units (slave) in cooperation with a central control unit (master) as a so-called “master-slave” arrangement. Control units may be arranged in the ventilation system, in the sedation by inhalation system (SIS), in the dispensing system, in the oxygenation system, in the switching unit or even in an external module.

These preferred embodiments of the system offer as advantages the fact that information of different systems can be combined with one another, and this also makes possible combinations of devices of different manufacturers and makes possible expansions of existing devices with additional devices or modules. The coordination and cooperation with one another is made possible by coordinated protocols in data exchange, for example, in a data network (LAN, WLAN).

In other preferred embodiments of the system, an individual control unit may be arranged in the non-central control system at least in the switching unit and/or an individual control unit may be arranged in the dispensing system and/or in the ventilation system and/or an external control unit may be arranged.

One or more of the individual control units and/or external control unit may be configured to control the switching unit and/or the dispensing system. The control may comprise here a coordination of the splitting and/or distribution of quantities of gas supplied and/or fed between the dispensing paths, i.e., between the purge gas dispensing path and the breathing gas dispensing path by means of the switching unit. In addition, the control may also comprise the manner of the dispensing by means of the dispensing unit, as well as a combined control of the switching unit and dispensing system, which control is coordinated with the operation of the system for supplying a gas or gas mixtures with feed of substances, for example, for carrying out an anesthesia by inhalation with combined feed of anesthetic gases into the breathing circuit and into the blood circulation of a patient.

These other preferred embodiments offer advantages concerning the coordination and control of the system in respect to the requirements imposed on computing power, storage requirement and response time, which are given for the individual functions.

Embodiments are possible to the effect that, for example, regulation processes with such high performance requirements in terms of time may occur for the dispensing directly in a control unit in the dispensing system, but, for example, a switching of the splitting of the quantities of gas into the oxygenation system and into the sedation by inhalation system can take place with moderate performance requirements in terms of time by means of the external control unit. Changes in this quantity splitting may also be carried out by a mobile terminal connected in a wireless manner as a special embodiment variant of an external control unit, e.g., a tablet computer, a smartphone or a mobile telephone.

In another preferred embodiment of the system for feeding substances, at least one of the control units can take into account data of the sedation by inhalation system (SIS), of the ventilation system (VS) and/or of the oxygenation system (OS), which data were provided at the time of the control of the switching unit. This other preferred embodiment offers the advantage that, for example, the information about changes that were made by the user, for example, in the mode of ventilation at the ventilator or about changes that were activated or initiated by the user shortly before can be taken into consideration in the control of the switching unit such that implementation of the initiated changes is waited for before a change in state is carried out by the switching unit. This also applies to initiated changes at the oxygenation system in respect to the control of the switching unit. Furthermore, possible alarms from the ventilator, ventilation system (VS), the sedation by inhalation system (SIS) and the oxygenation system can be taken into consideration for the control of the switching unit, for example, in such a manner that only certain changes are possible in the operating state of the switching unit in the presence of alarms.

The controller, control unit or the individual control units is/are configured to control the switching unit as well as the dispensing system. The control unit may, moreover, be configured to control the ventilator, the ventilation system, the sedation by inhalation system, the dispensing system, as well as the oxygenation system. The control unit may be arranged in this case as a functional element or control module in or at the ventilator, at the ventilation system, at the sedation by inhalation system, at the dispensing system, at the sedation by inhalation system (SIS) and at the oxygenation system or it may be associated with the ventilation system, with the sedation by inhalation system, with the dispensing system and with the oxygenation system.

The controller, control unit or the individual control units provides/provide many different functions for the operation of the system according to the present invention. A memory (RAM, ROM), which is configured to store a program code, is advantageously provided with the controller, such as in the control unit. The running of the program code is coordinated by a microcontroller arranged as an essential element in the control unit or by another embodiment of computing elements (FPGA, ASIC, μP, μC, GAL). The control unit and/or the individual control units is/are configured, prepared and intended to coordinate the operation of the system and/or the interaction of the ventilation system, sedation by inhalation system, dispensing system, oxygenation system and switching unit and other components and systems and to carry out the comparison operations, computing operations, storage and data organization of the data sets, actuations of actuators and sensors, measured data acquisition from sensors, data and information processing, as well as information and data provision, which are necessary in the process, to components in the interior of the system and to the outside of the system.

According to the first aspect of the present invention, a switching, splitting or distribution of quantities of gas takes place according to the present invention by means of the switching unit between the breathing gas connection system, especially the connection element (Y-piece) located close to the patient and the oxygenation system. With the switching, splitting or distribution the switching unit makes it possible for the substances provided to be able to be introduced both into the lungs of the patient for a sedation by inhalation and to be also able to enter into the blood circulation of the patient via the oxygenation system. This offers advantages, for example, when an inhalation treatment is desired with quantities of volatile, inhalable drugs or substances by means of the ventilator or by means of the ventilation system combined with the sedation by inhalation system and, if needed, at the same time or instead, also by means of the oxygenation system.

Another aspect of the present invention is formed by a gas splitting unit according to the present invention as an assembly unit at the connection element located close to the patient. This leads to a space-saving, compact arrangement with a small volume close to the access to the airways of the patient. A gas splitting unit according to the present invention forms a device for distributing and/or splitting partial quantities of breathing gas into the breathing circuit and into the blood circulation of the patient and it is formed by a common assembly unit with at least one switching unit, with a breathing gas dispensing path, with a connection element located close to the patient, by a gas removal port for removing partial quantities of breathing gas from the inhaled gas, and with a gas return port for inhaled gas. The switching unit, the breathing gas dispensing path, the connection element located close to the patient, the gas return port for inhaled gas and the gas removal port are configured and embodied as described in the context of the system according to the present invention for a feed of substances in the gas form according to the first aspect of the present invention. The common assembly unit has ports for the connection to the oxygenation system and to the dispensing system, to the ventilation system and to the patient. The breathing gas dispensing path and the gas return port may be formed in the gas splitting unit preferably and, for example, as internal lines. The purge gas dispensing path and the breathing gas connection system may be formed from the gas splitting unit preferably and, for example, as lines, e.g., in the form of ventilation tubes or tubings to the oxygenation system or to the patient and to the ventilation system. The supply line from the gas removal port to the dispensing system or to the system of sedation by inhalation (SIS) is likewise configured preferably and, for example, as tubing.

Thus, the essential advantage of the present invention with the first as well as the additional aspect of the present invention is that the administration of inhalable substances via the lungs of the patient can be split and can also be carried out extracorporeally into the blood circulation of the patient with the system for feeding substances in combination with a ventilation system, with a sedation by inhalation system (SIS) and with an oxygenation system.

In a preferred embodiment, the gas splitting unit may also comprise the reflection unit and/or the HME filter and or the additional filter element in the common assembly unit. The reflection unit and/or the HME filter are configured and embodied as described in the context of the system according to the present invention for feeding substances in the gas form according to the first aspect of the present invention.

In a preferred embodiment, the gas splitting unit and/or the connection element located close to the patient may have a port for a measured gas line, which is intended for connection to a process gas analysis unit.

In another preferred embodiment, a humidifying/heating system for breathing gas, which is intended to heat breathing gases, may be arranged in or at the gas splitting unit, in or at the switching unit, in or at the connection element located close to the patient and in/or at the breathing gas connection system.

In another preferred embodiment, a mixing chamber, which is intended for feeding exhaled gases of the patient by means of a waste gas line for exhaled gases, may be arranged in or at the gas splitting unit in another preferred embodiment. It becomes possible, at least partially, with the waste gas line that quantities of inhalable substances in the exhaled gas do not have to be disposed of continuously, but it is possible, instead, to recycle these quantities of inhalable substances. This leads to possibilities of savings concerning the inhalable substances, which brings with it advantages in terms of costs as well as in terms of reducing the emission of gases hazardous to the climate into the environment.

In another preferred embodiment, the waste gas line for exhaled gases, the gas splitting unit or the mixing chamber has an additional absorber unit, which is intended to remove carbon dioxide from the exhaled gas of the patient. This additional absorber unit makes it possible to remove carbon dioxide from the exhaled gas, so that a continuous reuse of returned residual quantities of inhalable substances in the exhaled gas can be made possible even independently from phases of respiration or the existing setting of the switching unit during the operation for splitting into the breathing gas dispensing path and the purge gas dispensing path.

In another preferred embodiment of the system—also including the gas splitting unit according to the additional aspect of the present invention—a device with a purge gas absorber unit and/or with an additional gas feed unit, configured, for example, as a blower for transporting purge gas in the oxygenation system or in the purge gas dispensing path, may be arranged and provided. The purge gas absorber unit removes the carbon dioxide present from the exhaled breathing gas, so that quantities of inhalable substances or volatile anesthetic not absorbed by the patient can be reduced for the treatment in a closed circuit after removal of carbon dioxide. The purge gas absorber unit contains a special kind of lime granules (soda lime), known as breathing lime usually consisting of calcium hydroxide [Ca(OH)₂] and/or sodium hydroxide [NaOH]. The carbon dioxide component is removed from the exhalation has by means of a chemical reaction while heat and water are released. A waste gas outlet (waste), via which used quantities of exhaled breathing gas can be sent for disposal, is provided in the ventilation system. This other preferred embodiment offers the advantage that purge gas processed by means of the purge gas absorber unit can be returned into the purge gas dispensing path and it can subsequently enter again the blood circulation of the patient at the membrane by means of the oxygenation connection system. Such an arrangement for recirculation may be called a so-called closed system. This purge gas absorber unit removes the carbon dioxide present, which is delivered from the blood circulation of the patient, from the purge gas, so that quantities of inhalable substances or volatile anesthetic not absorbed by the patient can be reused for the treatment in a closed circuit. The additional gas feed unit makes possible the circulation of the purge gas in a closed-circuit flow. Purge gas enriched with inhalable substances or volatile anesthetic can thus be prevented from having to be scavenged past the membrane as used gas by means of a waste gas outlet directly after a single-time flow and valuable substances can thus be reused for the further treatment. Such an additional gas feed unit may be arranged in combination with the additional purge gas absorber unit as a module, for example, as a plug-in module in the oxygenation system. The additional gas feed unit and the purge gas absorber unit may be configured together or also separately as independent units or modules, which may be connected, for example, as external modules to the oxygenation system.

The purge gas absorber unit is thus advantageously configured to remove a percentage of carbon dioxide from the purge gas, so that quantities of inhalable substances or volatile anesthetic (anesthetic) that are not introduced into the blood circulation at the membrane can be reused during the operation of the oxygenation system in the closed circuit after the removal of carbon dioxide. The breathing gas absorber unit contains a special kind of lime granules (soda lime) known as breathing lime, usually consisting of calcium hydroxide [Ca(OH)₂] and/or sodium hydroxide [NaOH]. The carbon dioxide component is removed from the purge gas by means of a chemical reaction while heat and water are released. A waste gas outlet (waste), via which quantities of used purge gas can be fed for disposal, is provided in the oxygenation system. All used quantities of gas are usually introduced into the infrastructure of the hospital by means of an anesthesia gas scavenging system (AGS: Anesthesia gas Scavenger) and are correspondingly disposed of properly. The quantities of gas fed to the process gas analysis units are usually fed after the analysis into the infrastructure of the hospital and are disposed of. However, these quantities of analyzed gas may also be reused and returned. Configurations with an open anesthesia gas scavenging (ORS: Open Reservoir Scavenger) are also possible; in this case, the used exhaled gas is filtered by means of an activated carbon collector and retained and the filtered exhaled gas is subsequently fed to the room air.

In an especially preferred embodiment of the system or of the gas splitting unit, a mixing chamber is arranged in or at the switching unit or in or at the gas splitting unit, and this mixing chamber is configured, in interaction with a feed line for exhaled gases from the ventilator to the switching unit, to reuse quantities or partial quantities of inhalable substances present in the exhaled gas, which are fed for disposal during the usual operation of the system for feeding substances via the exhalation valve of the ventilation system and via the waste gas outlet, during the further operation of the system and in the course of the treatment. The exhaled gas contains percentages of carbon dioxide, which can be removed from the exhaled gas by means of the purge gas absorber unit in a preferred embodiment of the oxygenation system.

It is possible in this manner, at least during certain time periods in the course of the ventilation treatment, to introduce partial quantities of exhaled gas containing residual percentages of inhalable substances—after removing the carbon dioxide present—into the blood circulation of the patient via the membrane of the oxygenation system, so that these partial quantities do not have to be disposed of.

In addition to the measuring system for the process gas analysis (PGA) for determining a gas concentration, which was already described above as a configuration of a preferred embodiment, an additional process gas analysis unit (PGA) or a plurality of process gas analysis units may be arranged in the system or associated with the system—also including the gas processing unit according to the additional aspect of the present invention—in preferred embodiments of the system. These process gas analysis units can provide data and/or information determined on the basis of the analysis for the control unit and/or for the control system or to individual control units. These other preferred embodiments offer the advantage that functions in terms of the monitoring of the dispensing of inhalable, volatile substances or anesthetics can be carried out continually during the operation and the effect of dispensing and/or changes in dispensing on the patient or on the status of the patient can be estimated on the basis of this monitoring. Some exemplary possibilities for arranging, assigning and using process gas analysis units (PGA) in the system will be explained in more detail below.

In special embodiments, the process gas analysis units (PGA) may be arranged in the system at individual components and they can thus be used independently from one another for the analysis. It is, however, also possible, and it is also covered as alternative additional embodiments in the sense of the present invention, that a process gas analysis unit arranged centrally in the system (PGA-C) with an additional switching unit and distribution control unit, configured, for example, as controllable and/or controlled valve arrays, forms a kind of an analysis center. Corresponding gas samples are made available now by the ventilation system, the oxygenation system and possibly also the sedation by inhalation system or the dispensing system or by the switching unit by means of the switching unit and the distribution control unit for the central process gas analysis unit (PGA-C) and they are then analyzed in series one after another as needed by the central process gas analysis unit (PGA-C).

The switching unit and the distribution control unit is equipped with means for switching, distributing and feeding gas samples to the individual component in the system, especially from the sedation by inhalation system (SIS), from the oxygenation system, from the oxygenation connection system, from the dispensing system, from the switching unit, from the gas splitting unit, from the connection element located close to the patient to the central process gas analysis unit (PGA-C) and to make it ready for the analysis and to coordinate the feeding of the gas samples. This other preferred embodiment offers the advantage that a respective process gas analysis unit (PGA) does not have to be arranged at each unit or at each module of the system. This can reduce the effort needed to build and operate components, such as sensor systems, power supply, interfaces and operating software, and to simplify the function and cooperation especially in case of an embodiment with a central control unit. The results of the analysis can then be made available to the individual control units or to the central control unit correspondingly in a non-central manner or centrally. In a special embodiment, a blood gas analysis unit may also be integrated in the process gas analysis unit (PGA-C) arranged centrally in the system. Such an additional process gas analysis unit may be arranged for an analysis of purge gases in or at the oxygenation system in or at the oxygenation connection system, or may be associated with the oxygenation system or with the oxygenation connection system. This additional process gas analysis unit (PGA-OS) can provide data determined on the basis of the analysis for the control unit and/or for an individual control unit. Knowledge of concentrations of carbon dioxide and/or oxygen in the purge gas is relevant for carrying out an extracorporeal membrane oxygenation.

A special manner of configuration of the process gas analysis unit may be configured in a preferred embodiment as an embodiment of a blood gas analysis unit (BGA) for the analysis of quantities of blood. This blood gas analysis unit according to this preferred embodiment may be arranged in or at the oxygenation system or in or at the oxygenation connection system, or may be associated with the oxygenation system or with the oxygenation connection system. The blood gas analysis unit (BGA) makes possible an analysis of the gases or gas mixtures dissolved in the blood of the patient, so that the blood gas analysis unit (BGA) provides data, for example, concerning a gas distribution (partial pressure) of O₂ (oxygen), CO₂ (carbon dioxide) as well as the pH value and the acid-base balance in the blood. The blood gas analysis unit can provide data determined on the basis of the analysis for the control unit and/or for an individual control unit.

Knowledge of these values may often be of interest or relevant for an evaluation of the effect of anesthesia, ventilation and/or extracorporeal membrane oxygenation. This other preferred embodiment offers the advantage of using the knowledge of the gas distribution (partial pressure) of O₂ (oxygen) and CO₂ (carbon dioxide) to monitor the control of the oxygenation system. In addition, meaningful information can be obtained for the user in respect to the general condition of the patient and concerning the performance of the treatment by means of the values obtained concerning the acid-base balance and the pH value of the blood. In addition, this blood gas analysis unit (BGA) can also check and/or monitor the function of the oxygenation system (quality of the oxygenator) in the course of the use. Indications on the current status as well as possible future changes in the status or changes in the properties of the oxygenator or membrane can thus be given to the user in time. The function of the oxygenation system may be impaired, for example, by coagulation effects (coagulation, clotting). Such a blood gas analysis unit (BGA) may be arranged in combination with the process gas analysis unit (PGA-OS) as a module, for example, as a kind of plug-in module in the oxygenation system. The blood gas analysis unit (BGA) and the process gas analysis unit (PGA-OS) may also be configured together or separately as independent units or modules, which may be connected as external modules to the oxygenation system. This combination and configuration as a module, especially and, for example, as a plug-in module, offers the advantage that the oxygenation system may optionally be equipped with modules and adapted to the situation, so that the oxygenation system can be set up with modules for blood gas analysis (BGA) and/or process gas analysis before use. Such a process gas analysis unit can thus be arranged for an analysis of breathing gases in or at the sedation by inhalation system (SIS) or in or at the breathing gas connection system or it may be associated with the sedation by inhalation system (SIS) or with the breathing gas connection system. This process gas analysis unit (PGA-SIS) may correspond here to the measuring system for process gas analysis for determining a gas concentration, which was mentioned before within the framework of the description of the dispensing system, or it may have a similar or similarly acting configuration. This process gas analysis unit (PGA-SIS) can provide data determined on the basis of the analysis for the control unit and/or for an individual control unit. The concentrations of certain gases, whose knowledge is relevant for carrying out a ventilation or anesthesia, can be determined in the breathing gas by means of the process gas analysis unit.

Knowledge of concentrations of carbon dioxide and oxygen in the breathing gas is relevant for carrying out a ventilation, just as well as for carrying out an anesthesia. Furthermore, knowledge of concentrations of gases or inhalable substances, substances for sedation by inhalation or anesthetics, for example, halothane, isoflurane, desflurane, sevoflurane or ether, may be relevant.

In a preferred embodiment of the system, a process gas analysis unit is arranged for an analysis in or at the dispensing system or is associated with the dispensing system. This process gas analysis unit (PGA-DS) may correspond here to the measuring system for a process gas analysis for determining a gas concentration, which was mentioned above within the framework of the description of the dispensing system, or may have a similar or similarly acting configuration. This process gas analysis unit (PGA-DS) may carry out—similarly to the process gas analysis unit, which is arranged for the analysis at the ventilation system—a gas analysis concerning the concentrations of certain gases. Concentrations of gases (oxygen, nitrous oxide, or inhalable substances, substances for sedation by inhalation or anesthetics, for example, halothane, isoflurane, desflurane, sevoflurane or ether, as well as oxygen, nitrous oxide (N₂O), nitrous oxide, Heliox, nitrogen monoxide) can be determined in this manner and this process gas analysis unit (PGA-DS) can thus make data determined on the basis of this analysis available to the control unit and/or to an individual control unit. This other preferred embodiment offers the advantage that the composition in the breathing gas is known in terms of the concentrations of substances, anesthetics, oxygen, nitrous oxide and other gases continually during the operation and a control and monitoring, as well as a regulation of the dispensing of gases is made possible by the control unit in the dispensing system itself or in a central control unit.

A process gas analysis unit is arranged in or at the switching unit or is associated with the switching unit for an analysis in a preferred embodiment of the system. Similarly to the process gas analysis unit, which is arranged for the analysis at the dispensing system, this process gas analysis unit (PGA-SU), can provide a gas analysis in terms of concentrations of gases or inhalable substances, substances for sedation by inhalation or anesthetics, for example, halothane, isoflurane, desflurane, sevoflurane or ether, as well as oxygen, and make data determined on the basis of this analysis available to the controller—the central control unit and/or to an individual control unit.

This other preferred embodiment offers the advantage that the composition in the breathing gas is known during the operation of the system for feeding substances and a control is made possible concerning the splitting of the enriched gas into the breathing gas dispensing path and into the purge gas dispensing path, including data on concentrations in the controller—a control unit in the switching unit, a control unit in the dispensing system or in a central control unit.

The provision of data and/or information in the system between the process gas analysis units, control unit, individual control units, configured, for example, as control modules, may take place by means of data lines or data links. The data lines or data links are preferably configured as a wired or wireless data network (Ethernet, LAN, WLAN, Bluetooth, PAN) or bus system (CAN, LON), which has data nodes for data coordination (switch, hub, router), on the one hand, as well as components (database, server, router, access point) for data storage and data distribution. For example, a databank system for organizing patient data in the form of a patient data management system (PMS) may be connected to the data network, which receives, in addition to diagnoses and treatment information belonging to patients, also the data and/or measured values of the process gas analysis units, which pertain to these patients, stores these as data sets and organizes the access thereto. The data network or bus system can also organize as a central element of the system the interaction of the individual control units with a central control unit, so that at least some of the components of the system, for example, the ventilation system, the sedation by inhalation system (SIS), the oxygenation system, the dispensing system, the switching unit, control units, individual control units, control modules, process gas analysis units, are connected to one another by means of the data network or bus system and can interact in a coordinated manner. Changes in the carrying out of the treatment with ventilation, extracorporeal oxygenation and decarboxylation can then advantageously be displayed in a combined form with patient data, diagnostic data, for example, ECG, EIT, laboratory data, for example, for blood, urine, cerebrospinal fluid, breathing gases or blood gases in a common display on a display unit integrated in the data network, which makes the effect of changes in the treatment visible to the user in a timely manner and makes it possible to check the effect.

Another preferred embodiment of a data network and/or network linking system, which is configured and intended to provide and to coordinate data in the system, in the control unit or in the individual control units, in the blood gas analysis unit, in the process gas analysis units, in the ventilation system, in the sedation by inhalation system (SIS), in the oxygenation system, in the switching unit, in the dispensing system or in other components, can be formed with data lines or data links, wired or wireless data network (Ethernet, LAN, WLAN, Bluetooth, PAN) or bus system (CAN, LON), data nodes for data coordination (switch, hub, router), components (database, server, router, access point) for data storage and data processing.

In a preferred embodiment of the system—also with inclusion of the gas splitting unit according to the additional aspect of the invention—a physiological patient monitoring (PPM) system may be arranged in or associated with the system for feeding substances. This other preferred embodiment offers the advantage that the effect of the administration of volatile substances and/or drugs and/or anesthetics on the status of the patient is monitored by measurement on the basis of physiological measured variables, such as oxygen saturation in the blood (SPO₂), carbon dioxide concentration during and at the end of the phase of exhalation (end-tidal carbon dioxide concentration, etCO₂), heart rate, blood pressure, and body temperature. The user can infer from these measured variables the current situation of the treatment in terms of both the blood gas exchange in the lungs and the extracorporeal blood gas exchange in respect to the removal of carbon dioxide and the feed of oxygen. In addition, it is possible to use the oxygen saturation in the blood (SPO₂) as a controlled variable for the dispensing of oxygen in the dispensing system, and, furthermore, the splitting of quantities of gas to the ventilation system and to the sedation by inhalation system (SIS) and to the oxygenation system can thus also be controlled in the switching unit. The carbon dioxide concentration can be used as the basis for controlling the extracorporeal blood gas exchange by the oxygenation system, which can be carried out, for example, by adapting flow rates at the blood feed unit and/or flow rates of the purge gas.

In a preferred embodiment of the system for feeding substances, also including the gas splitting unit according to the additional aspect of the present invention, a heart and lung imaging and diagnostic system may be arranged in the system or associated with the system.

This other preferred embodiment offers the advantage that the state of the lungs, especially also changes (improvement, recovery, exacerbation) of the situation of the lungs, can be followed up during the treatment. Suitable imaging systems are, for example, ultrasound diagnostic procedures, electrical impedance tomography (EIT), computed tomography (CT), X-ray, magnetic resonance imaging (MRI). Especially electrical impedance tomography (EIT) should be emphasized in this connection, because—contrary to the other four systems mentioned—it offers the possibility of a continuous imaging of the lungs, thorax and heart. Global and/or regional changes in the state of the lungs, in the type of ventilation of the lungs with possible regional hyperdistensions and collapses can thus be visualized. Changes in the type of ventilation by the ventilation system and in the manner of the combined use with the oxygenation system for the extracorporeal blood gas exchange are thus visible for the user by imaging in a timely manner and can be checked.

An especially preferred embodiment of the system—also with inclusion of the gas splitting unit according to the additional aspect of the present invention—makes possible by means of a provision of data a data exchange within the system with components of the system and with the data network or network linking system. A data exchange of the ventilation system, oxygenation system, sedation by inhalation system, dispensing system, switching unit, control units, process gas analysis units, blood gas analysis units, a heart and lung imaging and diagnostic system or a physiological patient monitoring (PPM) system with one another or with the data network or with the network linking system can now be made possible. The control unit of the switching unit, the control unit of the dispensing system or the individual control units in the system can thus be enabled to control and/or to coordinate the switching unit and or the dispensing unit. This other preferred embodiment offers the advantage that the aforementioned advantages of the possibilities of controlling and checking effects and interactions of the ventilation system, sedation by inhalation system, dispensing system, switching unit, and oxygenation system can be made available for the user such that they are combined with one another. The data exchange makes it possible to compare and to combine data in a chronological relation with one another and to display and to document the trend of the treatment in its entirety.

In another preferred embodiment, the control unit in the dispensing system may be configured to control the quantity of inhalable substances as a function of the data provided in the data network or network linking system and/or to control the quantity of inhalable substances as a function of the data provided by one of the control units. For example, the addition of the quantities of dispensed inhalable substance by the dispensing system can thus be carried out as a function of determined blood gas measured values, for example, the oxygen or carbon dioxide partial pressure in the blood, the acid-base balance or the pH value of the blood, determined by the blood gas analysis units (BGA), of measured values of the process gas analysis units (PGA), for example, oxygen and carbon dioxide concentrations in the breathing gas or of measured values of the physiological patient monitoring (PPM), which indicate states or situations of the cardiovascular system, for example, blood pressure, heart rate, ECG.

In other preferred embodiments of the system or of the gas splitting unit, the control unit controlling the switching unit may be configured to control the distribution and/or the splitting of the quantity of inhalable substances into the purge gas dispensing path to the oxygenation system and into the breathing gas dispensing path to the connection element located close to the patient or to the reflection unit as a function of the data provided in the data network or network linking system and/or as a function of the data provided by the controller, such as one of the control units. In particular, the controller—the control unit can coordinate or control the distribution and splitting of the partial quantities of breathing gas enriched with inhalable substances into the lungs of the patient or via the oxygenation system into the blood circulation of the patient on the basis of data that indicate a current state of the lungs of the patient and are provided, for example, by a heart and lung imaging and diagnostic system in the data network or network linking system. Thus, possible changes in the state of the lungs can be made visible continuously and in a timely manner during the treatment with EIT diagnostic systems (EIT system). Effects of the ventilation and of the manner of the combined use with the oxygenation system are thus visible to the user in a timely manner and they can be checked. If, for example, data of an EIT system, which indicate a current state of a ventilation situation of the lungs or changes, or a trend of the ventilation situation of the lungs of the patient, are made available in the network, the control unit of the switching unit can control based on this the distribution of the quantities of inhalable substances and/or of quantities of oxygen into the blood circulation or into the breathing circuit of the patient.

In case of a worsening ventilation situation, i.e., in a situation determined with the EIT system, in which lung regions are either not ventilated sufficiently any longer (ventilation) nor are they perfused sufficiently (perfusion) any longer or are neither ventilated sufficiently nor are perfused sufficiently, the control unit can prompt, for example, the switching unit to carry out the distribution of the breathing gas enriched with inhalable substances and possibly with oxygen between the breathing gas dispensing path and the purge gas dispensing path with an increase in the partial quantity of breathing gas into the purge gas dispensing path. In case of an improvement of the situation of the lungs of the patient, determined by means of the EIT system, which is, for example, a consequence of a recuperation or recovery of the lungs of the patient in the course of the treatment, the control unit can prompt the switching unit to carry out the distribution of the breathing gas enriched with inhalable substances and possibly with oxygen between the breathing gas dispensing path and the purge gas dispensing path with an increase in the partial quantity of breathing gas into the breathing gas dispensing path.

The present invention will now be explained in greater detail by means of the following figures and the corresponding descriptions of the figures without limitation of the general inventive idea. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first system for feeding inhalable substances;

FIG. 2 is a schematic view of a second system for feeding inhalable substances, and

FIG. 3 is a schematic view of a third system for feeding inhalable substances.

### DESCRIPTION OF PREFERRED EMBODIMENTS

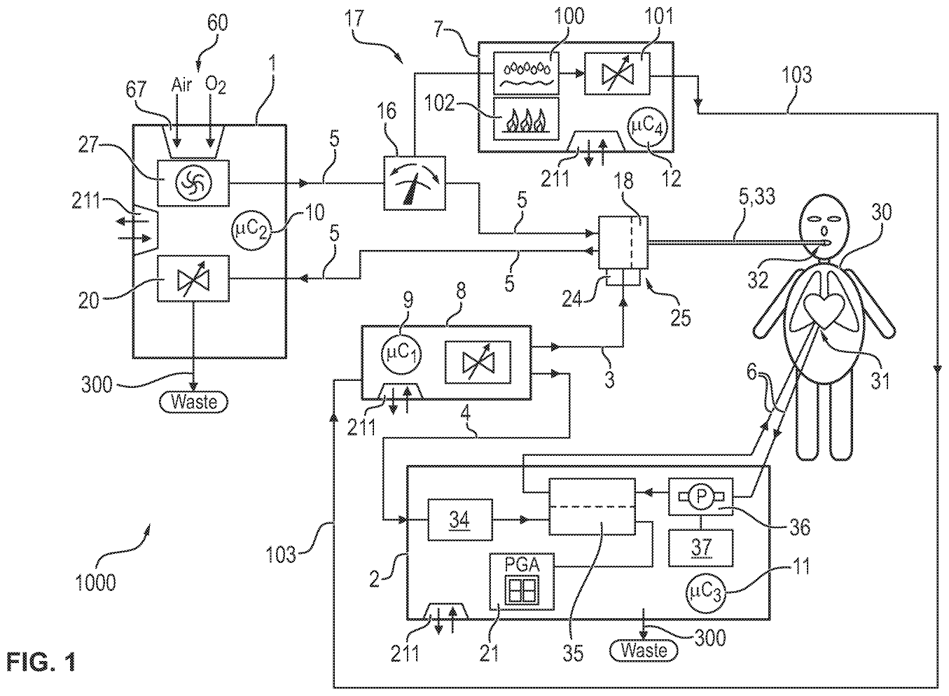

Referring to the drawings, FIG. 1 shows in a schematic view a patient 30 and a system 1000 for ventilation with oxygenation and decarboxylation with the essential principal components: Ventilator as a ventilation system 1, oxygenation system 2, breathing gas dispensing path 3, purge gas dispensing path 4, breathing gas connection system 5, oxygenation connection system 6, dispensing system 7, switching unit 8, gas removal port 16, gas return port 24, patient connection system comprising a connection element 25 located close to the patient, reflection unit 18, feed line 103 and a controller, comprising at least one control unit 9, provided and configured for controlling the dispensing system 7.

The patient 30 is connected fluidically to the ventilation system 1 for feeding and removing breathing gases by means of the breathing gas connection system 5 and of the connection element 25 located close to the patient, by means of an endotracheal tube 33 and of an airway access 32. A nasal mask or a tracheostoma may also be used as an alternative to the endotracheal tube 33. The sedation by inhalation system (SIS) 17 is formed essentially by the dispensing system 7, the gas removal port 16 for removing partial quantities of breathing gas from the inhaled gas, the reflection unit 18 and the gas return port 24 for returning the partial quantities of breathing gas from the switching unit 8 to the reflection unit 18. A partial quantity of breathing gas is made available and fed to the sedation by inhalation system (SIS) 17 from the ventilator 1 by means of the breathing gas connection system 5 and the gas removal port 16. An additional quantity of breathing gas, not enriched with inhalable substances, is fed directly to the airways 32 of the patient 30 via an endotracheal tube 33, the nasal mask or the tracheostoma by means of an additional component/additional path of the breathing gas connection system 5. The partial quantity of breathing gas, enriched with inhalable substances, reaches the switching unit 8 from the dispensing system 7 by means of the feed line 103. The partial quantity of breathing gas enriched with inhalable substances is fed from the switching unit 8 by means of the breathing gas dispensing path 3 to the airways 32 of the patient 30 via the gas return port 24 at the connection element 25 located close to the patient. The partial quantity enriched with inhalable substances is fed to the oxygenation system 2 from the switching unit 8 by means of the purge gas dispensing path 4. The switching unit 8 makes it possible to split and/or distribute partial quantities of gas enriched with inhalable substances into the breathing gas dispensing path 3 and into the purge gas dispensing path 4. Quantities of blood enriched with inhalable substances are fed into the blood circulation of the patient 30 from the oxygenation system 2 by means a purge gas flowing in the purge gas dispensing path 4 via the oxygenation connection system 6 and an invasive fluid access 31. A gas-to-blood exchange takes place in the oxygenation system 2 at a membrane 35 arranged in the oxygenation system 2. Quantities of gas enriched with oxygen O₂ and with the inhalable substances from the oxygenation connection system 6 reach the blood circulation of the patient 30 from the oxygenation connection system 6 while flowing around the membrane 35, and quantities of carbon dioxide CO₂ from the blood circulation of the patient 30 enter at the same time into the purge gas dispensing path 4. The ventilation system 1 is configured to supply breathing gases to the patient 30.

In the usual configuration, the ventilation system 1 is a part of a ventilator. Ventilation systems 1 for ventilators usually have means for supplying, feeding and removing breathing gases and substances to and from the patient, e.g., gas mixing devices 67 and gas feed devices 27, for example, a gas mixer and at least one gas feed unit (blower, piston drive, valve array), as well as gas feed devices, such as a gas port 60 for feeding gases, for example, air and oxygen, the breathing gas connection system 5, configured, for example, in the form of an inspiratory ventilation tube and often also of an expiratory ventilation tube and the connection element 25 located close to the patient, the so-called Y-piece, for connecting the ventilation tubes to the endotracheal tube 33, breathing mask or tracheostoma. Furthermore, a ventilation system 1 has an exhalation valve (exhalation valve) 20, through which exhaled gases returned via the expiratory ventilation tube of the breathing gas connection system 5 to the ventilator 1 can enter into the environment with the exhalation by the patient 30 via a waste gas outlet 300 or can be collected or removed by means of a system for collecting and removing quantities of used gas. In addition, alternative connection elements 25 located close to the patient are known as well, which also comprise an exhalation valve located close to the patient. In addition to the ventilation system 1, ventilators also have, in the usual configuration, elements, especially sensor systems, for detecting given and/or set pressures, flow rates and other operating parameters of a mechanical ventilation by measurement with a feed of gases and gas mixtures. At least the following parameters, such as inspiratory and expiratory ventilation pressures, ventilation rate, inhalation to exhalation ratio, upper and lower pressure limits, flow rate upper and lower limits, volume upper and lower limits and gas concentrations are set by a control unit 10 and/or monitored by means of the sensor system for a process of a mechanical ventilation. This sensor system is not shown in the view 1000 in this FIG. 1 for the sake of clarity. The dispensing system 7 is configured by means of a control unit 12 and a dispensing element 101 for an automated dispensing for dispensing a predefined quantity of substances and/or volatile anesthetic to a partial quantity of inhaled gas into the feed line 103 from a reservoir 100 containing inhalable substances and/or volatile anesthetic.

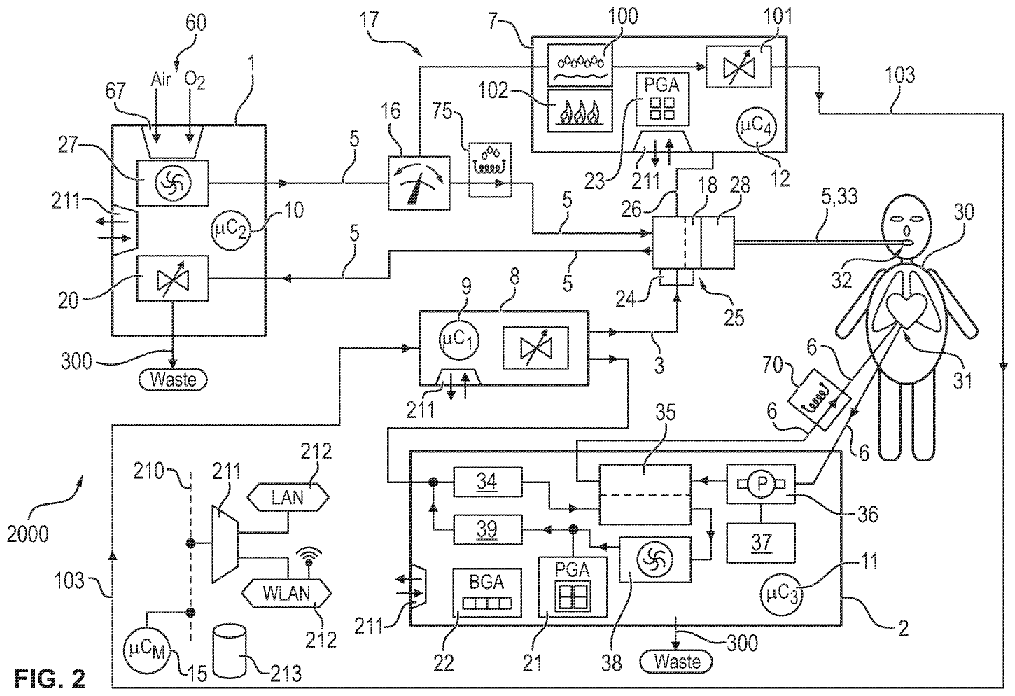

An anesthetic heater 102 may be activated by the control unit 12 in order to convert inhalable substances present in the liquid form in the reservoir 100 into a gas state of aggregation. An alternative embodiment variant for a manual dispensing or gas mixing would be an arrangement of so-called flow tubes, which can make possible the mixing of gases and/or the dispensing of inhalable substances or anesthetics by the interaction of needle valves and floating body flowmeters arranged in a rising tube. The switching unit 8 is configured by means of the control unit 9 to distribute or split the quantity of gases enriched with inhalable substances into the feed line 103 to the oxygenation system 2 or to the connection element 25 located close to the patient. The dispensing system 7 and the switching unit 8 are shown in this FIG. 1 as separate units, but the switching unit 8 may also be configured as an assembly unit of the dispensing system 7 in embodiments used in practice. The connection element 25 located close to the patient and the reflection unit 18 are shown in this FIG. 1 together with the gas return port 24 as a common unit. In embodiments used in practice, the connection element 25 located close to the patient, the reflection unit 18 and the gas return port 24 may also be configured as separate units. The connection element 25 located close to the patient, the reflection unit 18, and the gas return port 24 are shown in this FIG. 1 separately from the gas removal port 16. The connection element 25 located close to the patient, the reflection unit 18, the gas return port 24 and the gas removal port 16 may be configured in embodiments used in practice as a common assembly unit, for example, integrated into the connection element 25 located close to the patient. The control units 9, 10, 11, 12 may have a modular configuration or may be configured as a common control unit, and they may also form a central control unit 15 (FIG. 2) of the system 1000 or of the system 2000 (FIG. 2). Mixing of gases supplied by means of a gas port 60 takes place in the ventilation system 1 by means of the gas mixer 67. The gases oxygen and medical air are fed to the gas port 60, mostly by means of a central gas supply unit (GS). A quantity of breathing gas flows from the ventilation system 1 via the breathing gas connection system 5 to the patient 30. A partial quantity of breathing gas is removed from the breathing gas connection system 5 via the gas removal port 16 and is fed to the dispensing system 7.

Control of a gas feed unit 27 or of a piston drive, which can be used alternatively, is carried out in the ventilation system 1 by means of a control unit 10 in order to cause the breathing gas to flow to the patient 30 as well as to remove used breathing gases from the patient 30. The control unit 10 controls the course of the ventilation with inspiratory and expiratory ventilation pressures, tidal volumes, flow rates and other ventilation settings by means of an exhalation valve (exhalation valve) 20 and the gas feed unit 27. The breathing gas connection system 5 comprises an inhalation ventilation tube for feeding the breathing gas and an exhalation ventilation tube for removing the used exhaled gases of the patient 30, which are connected to one another by means of the connection element 25 located close to the patient, the so-called Y-piece, for connecting the patient 30. The setting and display elements, sensors for pressure and flow measurements, valves, nonreturn valves and other components, which are necessary for controlling the ventilation system 1 and for carrying out the ventilation, are not shown in this FIG. 1 for the sake of clarity. The patient 30 is connected to the oxygenation system 2 by means of the oxygenation connection system 6 for a supply with feeding and removing of quantities of blood into the blood circulation via an invasive fluid access 31. The patient 30 can be connected to the oxygenation system 2 via a fluid port 37, which is configured for a pumpless extracorporeal membrane oxygenation. The quantities of blood are transported to the patient 30 and away from the patient in such an embodiment by the pumping capacity of the heart of the patient himself. This configuration is called pumpless extracorporeal membrane oxygenation or pumpless extracorporeal lung assist (pECLA). However, the patient 30 is usually connected to the oxygenation system 2 by means of a blood feed unit 36, configured usually as a pump. The gas enriched with inhalable or volatile substances or anesthetic reaches a gas port 34 as a purge gas at the oxygenation system 2 by means of the purge gas dispensing path 4 from the switching unit 8. The oxygenation system 2 controls a quantity of flow and a flow velocity of the incoming flow of purge gas to the membrane 35 by means of a control unit 11. The membrane is configured to introduce oxygen from the purge gas into the blood and to remove carbon dioxide from the blood into the purge gas. A blood-to-gas exchange takes place in this manner outside the body (extracorporeally).

The setting and display elements, sensors for pressure and flow measurements, valves and other components, which are also necessary for controlling the oxygenation system 7 and for carrying out the extracorporeal enrichment with oxygen (oxygenation) and for the removal of carbon dioxide (decarboxylation), are not shown in this FIG. 1 for the sake of clarity. A process gas analysis unit 21 (PGA) associated with the oxygenation system 2 for the analysis of the gas composition of the purge gas is shown as another essential component of the system 1000. The process gas analysis unit 21 has, moreover, in addition to the measuring elements for determining gas concentrations, displaying and visualizing elements, as well as operating elements, not shown in this FIG. 1, which make reading and operation possible for a user. The process gas analysis unit 21 associated with the oxygenation system 2 is configured for an analysis of the gas composition of the purge gas. The purge gas is fed to the process gas analysis unit 21 and is analyzed in the process gas analysis unit 21 in order to monitor the ratios of carbon dioxide and oxygen at the membrane 35, and thus to determine the gas exchange and the transfer rate between blood circulation and purge gas and then to make possible an adequate control of oxygenation and decarboxylation for the patient by means of the control unit 11. Quantities of used gas are removed from the system 1000 from the oxygenation system 2 and from the ventilation system 1 via the waste gas outlet (waste) 300 via valve arrays, which are correspondingly provided for this purpose and are not shown in this FIG. 1. These quantities of used gas are usually introduced from the anesthesia device into the infrastructure of the hospital by means of an anesthetic gas removal system and are then properly disposed of there correspondingly. Depending on the splitting into the breathing circuit or into the blood circulation, the procedure is carried out with the system 1000 to feed substances related to oxygenation and decarboxylation by inhalation simultaneously with the performance of the ventilation with a gas-to blood exchange in the lungs of the patient 30 and/or extracorporeally with a gas-to-blood exchange at the membrane 35 of the oxygenation system 2. The ratio between the inhalable and extracorporeal administration of the inhalable substances can be set by the user by means of the switching unit 8. The measured values and status values of the process gas analysis unit (PGA) 21 of the oxygenation system 2 are available as a support for the user.

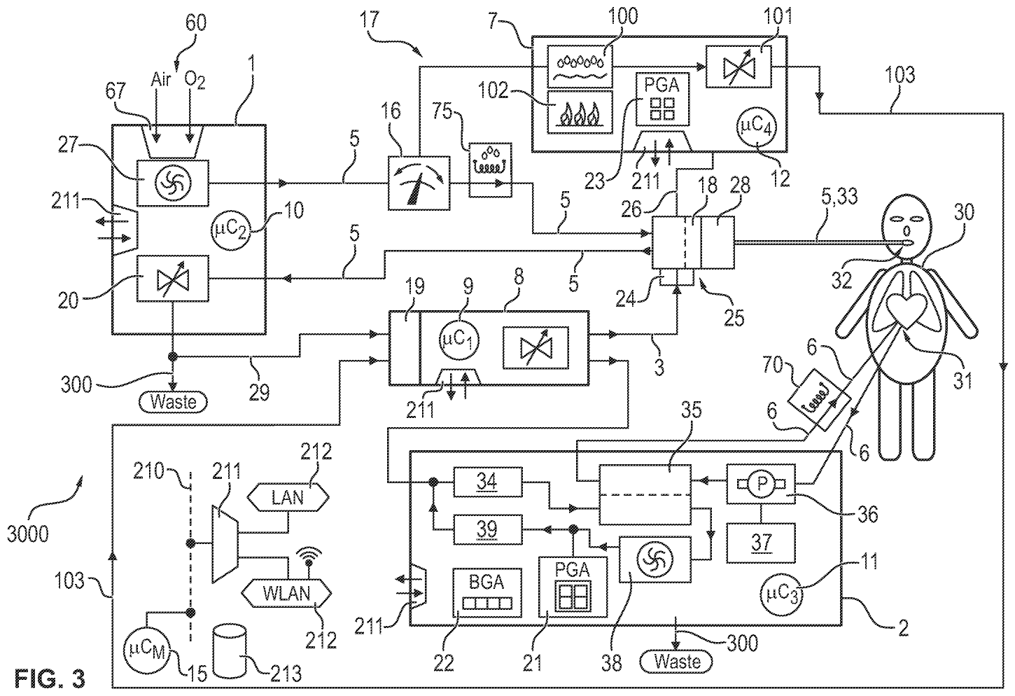

Data interfaces 211, which can make possible a unidirectional and/or bidirectional data exchange between the ventilation system 1, the oxygenation system 2, the dispensing system 7, the switching unit 8 and the sedation by inhalation system (SIS) 17, may be provided at the ventilation system 1, at the oxygenation system 2, at the dispensing system 7, and at the switching unit 8. Such a data exchange is preferably organized, initiated or coordinated in interaction and communication with the control units 9, 10, 11, 12 in the ventilation system 1, in the oxygenation system 2, in the sedation by inhalation system (SIS) 17, in the dispensing system 7, and in the switching unit 8. The data interfaces are connected to one another in a wired or wireless manner by means of data lines 210 (FIG. 2), which are not shown in the graphic representation in this FIG. 1 for the sake of clarity. An additional central control unit 15 (FIG. 2), not shown in this FIG. 1, may also be arranged in the system 1000, as well as in the systems 2000 (FIG. 2) and 3000 (FIG. 3), and it may be intended for coordinating the interaction in the system 1000 comprising the ventilation system 1, the oxygenation system 2, the sedation by inhalation system (SIS) 17, the dispensing system 7, the switching unit 8, optionally also with additional components 212, 213 (FIG. 2) (database, server, router, access point, hub) in a data network 212 (FIG. 2) (LAN, WLAN, Bluetooth, PAN, Ethernet) or network linking system.

FIG. 2 shows a system 2000 with possibilities of expanded configurations of the system 1000 for ventilation with oxygenation and decarboxylation according to FIG. 1.

Identical components in FIG. 1 and in FIG. 2 are designated with the same reference numbers in FIGS. 1 and 2.

In addition to the elements and components 1, 2, 3, 4, 5, 6, 7, 8, 9, 10, 11, 12, 16, 17, 18, 20, 21, 24, 25, 27, 30, 31, 32, 33, 34, 35, 36, 37, 60, 67, 100, 101, 102, 103, 211, and 300 shown in FIG. 1 and described for the system 1000 (FIG. 1), additional and features and components 15, 19, 22, 23, 26, 28, 38, 39, 70, 75, 210, 212, and 213 are present in the expanded system 2000 according to FIG. 2.

Thus, the expanded system 2000 has an additional gas feed unit (blower) 38 and a purge gas absorber unit (carbon dioxide absorber) 39 in the oxygenation system 2. Such an additional gas feed unit 38 may be arranged in combination with the purge gas absorber unit 39 as a module, for example, as a kind of plug-in module in the oxygenation system 2.

The additional gas feed unit 38, as well as the purge gas absorber unit 39, may also be configured together or separately as independent units or modules, which may be connected, for example, as external modules to the oxygenation system 2. Thus, the expanded system 200 shows a measured gas line (sample line) 26, which can be joined to the connection system element 25 located close to the patient or can be connected to this, and through which samples of the breathing gas given at the patient 30 can be fed to an additional process gas analysis unit 23 or blood gas analysis unit 23, so that the process gas analysis unit 23 or the blood gas analysis unit 23 is capable, based on measurement, of determining concentrations of oxygen, carbon dioxide, or inhalable substances, for example, anesthetics and of determining measured values, which indicate these concentrations and of making them available for the control of the system 2000. For the further analysis, the expanded system 2000 may have, in addition, a blood gas analysis unit (BGA) 22 for the analysis of blood gases in the blood of the patient 30 in the oxygenation system 2. A blood gas analysis provides, for example, information concerning a gas distribution (partial pressure) of O₂ (oxygen), CO₂ (carbon dioxide) as well as the pH value and the acid-base balance in the blood of the patient 30. Such a blood gas analysis unit 22 (BGA) may be arranged in combination with the process gas analysis unit (PGA) 21 as a module, for example, as a kind of plug-in module in the oxygenation system 2. The blood gas analysis unit 22 (BGA) and the process gas analysis unit (PGA) 21 may also be configured together or separately as independent units or modules, which may be connected, for example, as external modules to the oxygenation system 2. The switching unit 8 and the dispensing unit 7 may also be configured in a common assembly unit, so that the process gas analysis unit 23 or the blood gas analysis unit 23 may then also be arranged within the common assembly unit both in or at the dispensing unit 7 or in or at the switching unit 8. The configuration of a common assembly unit with arrangement of the process gas analysis unit or blood gas analysis unit is not shown in this FIG. 2 for the sake of clarity. The expanded system 2000 shows as an additional component a humidifying and/or heating system 75 for controlling the temperature of breathing gases in the breathing gas connection system 5.

FIG. 3 shows a system 3000 with expansions of the configurations of the systems 1000, 2000 for ventilation with oxygenation and decarboxylation according to FIG. 1 or 2. Identical components in FIGS. 1, 2, and 3 are designated by the same reference numbers in FIGS. 1, 2 and 3.

In addition to the elements and components 1, 2, 3, 4, 5, 6, 7, 8, 9, 10, 11, 12, 15, 16, 17, 18, 19, 20, 21, 22, 23, 24, 25, 26, 27, 28, 30, 31, 32, 33, 34, 35, 36, 37, 38, 39, 60, 67, 70, 75, 100, 101 102, 103, 210, 211, 212, 213 and 300, which are shown and described in FIG. 2 in connection with system 2000 (FIG. 2), additional components 19, 29 are present in the expanded system 3000 according to FIG. 3. Thus, the switching unit 8 has a mixing chamber 19. This mixing chamber 19 is configured and intended to receive at least partial quantities of the exhaled gases of the patient 30 in this mixing chamber 19 of the switching unit 8 by means of the waste gas line 29, instead of allowing these—as is shown in the embodiments according to FIGS. 1 and 2—to flow from the waste gas outlet 300 into the environment. These partial quantities of exhaled gases can then be sent from the mixing chamber 19 of the switching unit 8, together with the quantities of breathing gas fed by the dispensing system 7 by means of the feed line 103 and enriched with inhalable substances, to the switching unit 8 and then be fed via the purge gas connection path 4 to the oxygenation system 2. The concentration of carbon dioxide in the gas mixture is reduced by the purge gas absorber unit 39 in the oxygenation system 2 and the quantity of inhalable substances remaining after exhalation by the patient can again be fed via the oxygenation system 2 by means of the oxygenation connection system 6. It is possible in this manner, depending on the selected splitting of the quantities of gas containing inhalable substances between the oxygenation system 2 and the ventilation system 1, by the switching unit 1, to reuse partial quantities of the inhalable substances still present in the exhaled gas of the patient 30 in the oxygenation system for the extracorporeal gas exchange, at least during defined time periods in the course of the ventilation by the ventilator 1, instead of allowing them to flow into the environment via the waste gas outlet or instead of having to dispose of them by means of a scavenging or collection system (AGS: Anesthesia Gas Scavenger, ORS: Open Reservoir Scavenger). It thus becomes possible at least partially due to the waste gas line 29 not to have to send quantities of inhalable substances in the exhaled gas continuously for disposal, but it is possible, instead, to reuse these quantities of inhalable substances. In particular, if the switching unit 8 is set such that the distribution of inhaled gas enriched with inhalable substances flows to the oxygenation system 2 essentially via the purge gas dispensing path, an appreciable percentage of the residual quantities of inhalable substances present in the exhaled gas returned in the exhaled gas to the ventilator can then be reused in the oxygenation system 2.

If an optional additional absorber unit 68 is introduced into the waste gas line 29, into the mixing chamber 19 of the switching unit 8 or into the switching unit 8, it thus becomes possible to remove carbon dioxide from the exhaled gas, so that there is a possibility for the continuous reuse of the residual quantities of inhalable substances returned to the ventilator with the exhaled gas even independently from phases of respiration or from the particular setting of the splitting into the breathing gas dispensing path 3 and into the purge gas dispensing path 4 after the CO₂ removal, which setting is present during the operation.

Moreover, an additional process gas analysis unit (PGA) 23, arranged in the expanded systems 2000, 3000 according to FIGS. 2 and 3, for example, at the switching unit 8 or at the dispensing unit 7 is provided for the analysis of the gas 103 in the oxygenation dispensing path and/or in the breathing gas dispensing path 3, purge gas dispensing path 4 or in a path starting from the dispensing unit 7. Information concerning dispensing and the setting of the anesthetic dispensing 100, 101, 102 can thus be checked by measurement by means of concentration determination in the gas 103. Depending on the set splitting of the gas 103 into the breathing circuit or into the blood circulation to the switching unit 8, the gas in the breathing gas dispensing path 3 and the gas in the purge gas dispensing path 4 have different oxygen concentrations. The additional process gas analysis unit (PGA) 23 may be useful for monitoring this difference by measurement. In such a mode of operation, the anesthesia is carried out with the ventilation system 1 with the feed of volatile anesthetic as well as with the feed of additional substances, preferably volatile substances, by inhalation along with the performance of the ventilation with a gas-to-blood exchange directly in the lungs of the patient 30 or extracorporeally with a gas-to-blood exchange at the membrane 35 of the oxygenation system 2, depending on the desire of the user with different concentrations of oxygen in the breathing gas indirectly 5, 32, 33 to the lungs of the patient 3 and indirectly 6, 31 into the blood circulation of the patient 30.

The systems 1000, 2000, 3000 shown in FIGS. 1 through 3 may be connected for an interaction and for a common system operation by means of the data interfaces 211, data lines 210 in the data network 212 with the additional medical devices or systems, for example, with process gas analysis units (PGA) 20, 21, 23, blood gas analysis units (BGA) 22, the physiological patient monitoring (PPM) system 40 as well as with the heart and lung imaging and diagnostic system 50.

For example, the system 1000 and the expanded systems 2000, 3000 may thus have a physiological patient monitoring (PPM) system 40. Such a physiological patient monitoring system 40 has displays and visualizations of detected, determined, analyzed or calculated physiological measured data and/or parameters. These include, for example, measurement-based detections of ECG by means of ECG electrodes on the upper body of the patient and ECG cables, detection of an oxygen saturation (SPO₂), for example, on the finger of the patient 30, detection of a non-invasive blood pressure measured value obtained by means of a blood pressure cuff on the upper arm of the patient 30, detection of an invasive blood pressure measured value obtained by means of an invasive access point on the hand of the patient 30, as well as of a body temperature, e.g., a skin temperature or a body core temperature of the patient 30. Gas samples can be sent via an optional port for gas suctioning at the Y-piece 25 and/or via another measured gas line (not shown in detail in FIG. 2 and in FIG. 3) to a physiological patient monitoring system 40 and gas analyses may be carried out in this system, for example, concentrations of carbon dioxide and methane can be determined, or analyses of other components, for example, alcohols (ethanol) in the exhaled gas can be determined. Thus, the control unit 12 in the dispensing system 7 may be configured to control the quantity of inhalable substances 100 as a function of the data provided in the data network 212 or in the network linking system and/or as a function of the data provided by one of the control units 9, 10, 11, 15. For example, the addition of the quantities of dispensed inhalable substances 100 by the dispensing system 7 can be carried out as a function of an oxygen or carbon dioxide partial pressure in the blood, of the acid-base balance or the pH value of the blood, concentrations of oxygen and carbon dioxide in the breathing gas or the blood pressure, heart rate, and ECG. The system 1000 and the expanded systems 2000, 3000 may also have—not shown in detail in FIG. 2 and in FIG. 3—a heart and lung imaging and diagnostic system 50. Heart and lung imaging and diagnostic systems 50 are configured, for example, as devices for computed tomography (CT diagnostics), magnetic resonance imaging (MRI diagnostics), X-ray devices (X-ray diagnostics), electrical impedance tomography devices (EIT diagnostics, EIT system) or ultrasound diagnostic devices (US diagnostics, sonography, Doppler sonography). The heat and lung imaging and diagnostic system 50 can provide the user with valuable information on the pathological state or the state of recovery of the lungs of the patient 30.

Based on this, the user can configure the systems 1000, 2000, 3000 such as to place the main focus of feeding oxygen to the patient 30 by inhalation on the path via the lungs or by means of the extracorporeal membrane oxygenation (ECMO) invasively on the path via the blood circulation. Contrary to CT diagnostics, X-ray diagnostics, MRI diagnostics, US diagnostics, especially electrical impedance tomography devices (EIT diagnostics) make possible a continuous imaging of the lungs, thorax and heart. Thus, possible changes in the state of the lungs can be visualized continuously and in a timely manner during the treatment with systems 50 for EIT diagnostics (EIT system). Effects of the ventilation and of the manner in which the combined use with the oxygenation system is employed are thus visible to the user in a timely manner and they can be checked. If, for example, data of an EIT system 50 are provided in the network 212, which data indicate a trend in the ventilation situation of the lungs of the patient 30, the control unit 9 of the switching unit 8 can control the distribution of the quantities of inhalable substances 100 and/or of the quantities of oxygen into the blood circulation or into the breathing circuit of the patient 30 on the basis of these data. For example, in case of an exacerbation of the ventilation situation, i.e., when the EIT system 50 determines that lung regions are no longer ventilated sufficiently (ventilation) or are not perfused sufficiently (perfusion) any longer or that they are neither ventilated sufficiently nor are they perfused sufficiently any longer, the control unit 9 can prompt the switching unit 8 to carry out the distribution of the breathing gas enriched with inhalable substances 100 between the breathing gas dispensing path 3 and the purge gas dispensing path 4 such that it increases the partial quantity of breathing gas into the purge gas dispensing path 4. In case of an improvement of the situation of the lungs of the patient 30, which is determined by means of the EIT system 50, for example, as a consequence of a recuperation or recovery of the lungs of the patient 30 in the course of the treatment, the control unit 9 can prompt the switching unit 8 to carry out the distribution of the breathing gas enriched with inhalable substances 100 between the breathing gas dispensing path 3 and the purge gas dispensing path 4 by increasing the partial quantity of breathing gas into the breathing gas dispensing path 3.

While specific embodiments of the invention have been shown and described in detail to illustrate the application of the principles of the invention, it will be understood that the invention may be embodied otherwise without departing from such principles.

### LIST OF REFERENCE NUMBERS

1 Ventilation system (VS), ventilator
2 Oxygenation system (OS) (oxygenator)
3 Breathing gas dispensing path
4 Purge gas dispensing path
5 Breathing gas connection system
6 Oxygenation connection system
7 Dispensing system (DS)
8 Switching unit
9 Control unit, control module (μC1) of the switching unit
10 Control unit, control module (μC2) of the ventilator/ventilation system
11 Control unit, control module (μC3) of the oxygenation system (OS)
12 Control unit, control module (μC4) of the dispensing system (DS)
15 External control unit, external control module (μLCM)
16 Gas removal port for inspiratory breathing gas, inhaled gas
17 Sedation by inhalation system (SIS)
18 Reflection unit (CR), element for anesthetic gas recovery, anesthetic gas reflector
19 Mixing chamber at switching unit
20 Exhalation valve of the ventilator
21 Process gas analysis (PGA, PGA-OS) of the oxygenation system
22 Blood gas analysis (BGA) of the oxygenation system
23 Process gas analysis (PGA, PGA-DS, PGA-SIS) of the dispensing system
24 Gas return port for inspiratory breathing gas, inhaled gas
25 Patient connection system with connection element located close to the patient (Y-piece)
26 Measured gas line
27 Gas feed unit (blower, piston drive) in the ventilation system
28 Filter element for moisture recovery (HME filter)
29 Waste gas line for exhaled gas
30 Patient, living being
31 Invasive fluid access to the blood circulation of the patient
32 Airway access, access to the airways of the patient
33 Endotracheal tube, alternatively nasal mask or tracheostoma
34 Gas port at the oxygenation system
35 Membrane, blood<->gas exchange membrane, oxygenator membrane
36 Fluid port with blood feed unit (pump)
37 Fluid port for pumpless extracorporeal membrane oxygenation
38 Additional gas feed unit (blower) in or at the oxygenation system
39 Purge gas absorber unit, carbon dioxide absorber (CO₂ remove) in the oxygenation system
40 Physiological Patient Monitoring (PPM) system
50 Heart and lung imaging and diagnostic system
60 Gas port for feeding gases (oxygen, air) to the ventilator
67 Gas mixer for mixing gases (oxygen, air) in the ventilator
68 Additional absorber unit
70 Heating system for quantities of blood at the oxygenation connection system
71 Humidifying/heating system for breathing gas at the breathing gas connection system
100 Reservoir (anesthetic tank) for inhalable substances or anesthetics
101 Dispensing element
102 Anesthetic heater
103 Feed line for supplying the gas mixture to the switching unit 8
210 Data lines, data links, data nodes
211 Data interfaces, data nodes, data coordination (switch, hub, router)
212 Network linking system, data network (LAN, WLAN, Bluetooth, PAN, Ethernet)
213 Components in the data network (database, server, router, access point, hub)
300 Waste gas outlet (waste)
1000 System (FIG. 1)
2000 Expanded system (FIG. 2)
3000 Expanded system (FIG. 3)

What is claimed is:

1. A ventilating and oxygenating system for ventilating and oxygenating a patient, the ventilating and oxygenating system comprising:
a ventilation system configured with devices for supplying breathing gases to the patient with a breathing gas connection system connected to the ventilation system, wherein the breathing gas connection system is configured for a gas-carrying connection for a supply with feeding and removal of breathing gases to the patient;
a patient connection system comprising a patient connection element located adjacent to the patient, the patient connection system being connected to the breathing gas connection system;
an oxygenation system with an oxygenation connection system, wherein the oxygenation system comprises a membrane configured for a gas exchange with a blood circulation of the patient with a feed of a quantity of oxygen and of a quantity of inhalable and/or volatile substances into the blood circulation of the patient and for removing carbon dioxide from the blood circulation of the patient, and wherein the oxygenation system comprises devices for feeding and/or supplying a quantity of the purge gas to the membrane and wherein the oxygenation connection system is configured to supply the patient with quantities of blood enriched with the inhalable and/or volatile substances and with oxygen and remove quantities of blood enriched with carbon dioxide;
a sedation by inhalation system comprising a dispensing system configured to dispense the inhalable and/or volatile substances, a gas removal port, a reflection unit and a gas return port;
a breathing gas dispensing path;
a purge gas dispensing path;
a switching unit, wherein the switching unit is configured for splitting and/or distributing quantities of gas enriched with the inhalable and/or volatile substances into the breathing gas dispensing path and into the purge gas dispensing path, and is configured to feed and to supply a partial quantity of breathing gas enriched with the inhalable and/or volatile substances by means of the breathing gas dispensing path and by means of the connection element located adjacent to the patient to the airways of the patient and is configured to feed and to supply a partial quantity of breathing gas enriched with the inhalable and/or volatile substances to the oxygenation system by means of the purge gas dispensing path, wherein the breathing gas connection system is configured for feeding a partial quantity of breathing gas enriched with the inhalable and/or volatile substances from the switching unit via the connection element located adjacent to the patient to the patient and the breathing gas connection system is configured for feeding an additional partial quantity of breathing gas not enriched with inhalable and/or volatile substances from the ventilation system to the patient via the connection element located adjacent to the patient; and
a controller, comprising at least one control unit, configured for controlling the switching unit.

2. A ventilating and oxygenating system in accordance with claim 1, wherein the dispensing system is configured to dispense inhalable and/or volatile substances or volatile anesthetics.

3. A ventilating and oxygenating system in accordance with claim 1, wherein the gas return port, the reflection unit and the patient connection element system located adjacent to the patient are configured as one assembly unit.

4. A ventilating and oxygenating system in accordance with claim 1, wherein a filter element is arranged at the reflection unit.

5. A ventilating and oxygenating system in accordance with claim 4, wherein:
the filter element is configured as a heat and moisture exchanging (HME) filter for absorbing and releasing moisture; or
the filter element is configured as a filter for retaining germs, viruses or bacteria present in the breathing gas.

6. A ventilating and oxygenating system in accordance with claim 4, wherein the gas removal port and the gas return port are configured in a common assembly unit with the reflection unit and/or with the breathing gas connection system and/or with the connection element located adjacent to the patient and/or with the filter element.

7. A ventilating and oxygenating system in accordance with claim 4, wherein the gas removal port and the gas return port are configured in a common assembly unit with the reflection unit and/or with an exhalation valve located adjacent to the patient and/or with the breathing gas connection system and/or with the connection element located adjacent to the patient and/or with the filter element.

8. A ventilating and oxygenating system in accordance with claim 1, wherein the control unit is configured to control the dispensing system to control the dispensing of the inhalable and/or volatile substances on the basis of concentrations of inhalable and/or volatile substances determined at the connection element located adjacent to the patient, at the breathing gas connection system or at the reflection unit.

9. A system in accordance with claim 8, wherein the control unit is configured to control the dispensing system to control the dispensing of the inhalable and/or volatile substances on the basis of an end-tidal concentration of at least one inhalable substance or of at least one anesthetic.

10. A ventilating and oxygenating system in accordance with claim 1, wherein the dispensing system is configured as a part of at least one of:
the sedation by inhalation system;
the ventilation system; and
the breathing gas connection system.

11. A ventilating and oxygenating system in accordance with claim 1, wherein the switching unit is configured as a part of at least one of:
the sedation by inhalation system;
the ventilation system;
the breathing gas connection system;
the oxygenation connection system;
the purge gas dispensing path;
the purge gas dispensing path; and
the dispensing system.

12. A ventilating and oxygenating system in accordance with claim 1, further comprising a blood feed unit, for transporting quantities of blood to the patient and/or away from the patient, arranged in or at the oxygenation connection system and/or at the oxygenation system.

13. A gas splitting unit for a ventilating and oxygenating system for ventilating and oxygenating a patient, the gas splitting unit comprising:
a switching unit;
a breathing gas dispensing path;
a connection element located adjacent to the patient;
a gas removal port configured to remove partial quantities of breathing gas from inhalation gas;
a gas return port for inhalation gas; and
a purge gas dispensing path, wherein at least the switching unit, the breathing gas dispensing path, the connection element located adjacent to the patient, and the gas removal port, the gas return port form a common assembly unit.

14. A gas splitting unit in accordance with claim 13, wherein the gas splitting unit further comprises a reflection unit and/or a heat and moisture exchanging (HME) filter and/or an additional filter element in the common assembly unit.

15. A system in accordance with claim 1, wherein:
the switching unit, the breathing gas dispensing path, the connection element located adjacent to the patient, the gas removal port, which is configured to remove partial quantities of breathing gas from inhalation gas, the gas return port for inhalation gas, and the purge gas dispensing path, comprise a gas splitting unit;
at least the switching unit, the breathing gas dispensing path, the connection element located adjacent to the patient, and the gas removal port, the gas return port form a common assembly unit; and
a gas feed unit, configured to transporting purge gas, is arranged in the gas splitting unit, in the purge gas dispensing path or in the oxygenation system.

16. A system in accordance with claim 15, wherein a purge gas absorber unit for removing carbon dioxide from the purge gas is arranged in the purge gas dispensing path or in the oxygenation system.

17. A system in accordance with claim 15, further comprising a waste gas line extending from the ventilation system to the gas splitting unit or to the switching unit and configured to feed exhaled gases from the ventilation system to a mixing chamber arranged in or at the switching unit or in or at the gas splitting unit and the removal of carbon dioxide from the breathing gas.

18. A system in accordance with claim 15, wherein the controller is configured as a central control system or as a central control unit.

19. A system in accordance with claim 15, wherein:
the controller comprises the at least one control unit and further individual control units forming a non-central control system; and
at least one of the at least one control unit and further individual control units is arranged in the oxygenation system and in the ventilation system.

20. A system in accordance with claim 19, wherein:
at least one of the at least one control unit and further individual control units is arranged in the switching unit and/or is arranged in the dispensing system and/or an external control unit is arranged in the non-central control system;
one of the individual control units and/or the external control unit is configured as the at least one control unit to control the switching unit and/or the dispensing system.

21. A system in accordance with claim 15, wherein the controller takes into consideration respective provided data of the ventilation system and/or of the oxygenation system when controlling the switching unit.

22. A system in accordance with claim 15, further comprising a process gas analysis unit arranged in or at the oxygenation connection system or associated with the oxygenation system, or arranged in or at the oxygenation connection system, wherein the process gas analysis unit is configured to provide gas analysis data determined based on an analysis for the system or for the controller.

23. A system in accordance with claim 15, further comprising a process gas analysis unit, for an gas analysis, arranged in or at the patient connection element located adjacent to the patient, or connected to the gas splitting unit or to the connection element located adjacent to the patient by means of a measured gas line, or arranged in or at the sedation by inhalation system or is associated with the sedation by inhalation system, wherein the process gas analysis unit is configured to provide gas analysis data determined on the basis of the analysis for the sedation by inhalation system, for the system and/or for the controller.

24. A system in accordance with claim 23, wherein:
a central process gas analysis unit is arranged in the system or is associated with the system;
the central process gas analysis unit is configured, together with a switching and distribution control unit, to carry out analyses of gas samples of the sedation by inhalation system, of the breathing gas connection system, of the connection element located adjacent to the patient, of the oxygenation system, of the oxygenation connection system, of the dispensing system or of the switching unit and/or to provide data determined on the basis of the analyses for the system and/or for the controller.

25. A system in accordance with claim 23, wherein:
a blood gas analysis unit for a blood analysis is arranged in or at the oxygenation system or at the oxygenation connection system or is associated with the oxygenation system or with the oxygenation connection system; and
the blood gas analysis unit is configured to provide data determined on the basis of the analysis for the oxygenation system, for the system and/or for the controller.

26. A system in accordance with claim 23, wherein:
a process gas analysis unit for a gas analysis is arranged in or is associated with the switching unit or the dispensing system; and
the process gas analysis unit is configured to provide gas analysis data determined on the basis of the analysis for the switching unit, for the dispensing system, for the system and/or for the controller.

27. A system in accordance with claim 15, wherein a humidifying/heating system for breathing gas is arranged for heating breathing gases in or at one or more of the gas splitting unit, the switching unit, the connection element located adjacent to the patient, and the breathing gas connection system.

28. A system in accordance with claim 15, wherein:
a data network is arranged in or at the system or is associated with the system;
the data network is configured to provide data to at least one of the system, the controller, the blood gas analysis unit, the process gas analysis units, the ventilation system, the oxygenation system, the switching unit, the dispensing system, and the sedation by inhalation system to enable the controller to control and/or to coordinate the switching unit and/or the dispensing unit.

29. A system in accordance with claim 15, further comprising a physiological patient monitoring system arranged in or connected to the system, wherein the physiological patient monitoring system is configured to provide physiological data for the system, for the ventilation system, for the oxygenation system, for the dispensing system, for the switching unit, for the controller and/or for a data network.

30. A system in accordance with claim 15, further comprising a heart and lung imaging and diagnostic system arranged in or connected to the system, wherein the heart and lung imaging and diagnostic system is configured to provide data for the system, for the ventilation system, for the oxygenation system, for the dispensing system, for the switching unit, for a physiological patient monitoring system, for the controller and/or for a data network.

31. A system in accordance with claim 15, wherein the system is configured to provide data with a data network in a data exchange.

32. A system in accordance with claim 15, wherein the controller is configured to control a quantity of inhalable and/or volatile substances as a function of data provided in a data network and/or as a function of a data provided by the controller.

33. A system in accordance with claim 15, wherein the controller is configured to control a distribution and/or a splitting of a quantity of inhalable and/or volatile substances into the purge dispensing path to the oxygenation system and into the breathing gas dispensing path to the connection element located adjacent to the patient or to the reflection unit as a function of the data provided in a data network and/or as a function of the data provided by the controller.
